(19) United States
(12) Patent Application Publication
(10) Pub. No.: US 2004/0241800 A1
(43) Pub. Date: Dec. 2, 2004

(54) Vascular endothelial growth factor dimers

(76) Inventors: Rodney Alan Jue, Castro Valley, CA (US); Ute Schellenberger, Palo Alto, CA (US); Peter A. Stathis, Menlo Park, CA (US); Peter Isadore Adriaenssens, Mountain View, CA (US); Judith A. Abraham, San Jose, CA (US); Patricia Ann Baldwin, Los Altos, CA (US); N. Stephen Pollitt, Los Altos, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/749,703

(22) Filed: Dec. 31, 2003

Related U.S. Application Data
(62) Division of application No. 09/575,199, filed on May 18, 2000.
(60) Provisional application No. 60/177,407, filed on Jan. 20, 2000.
(60) Provisional application No. 60/135,312, filed on May 20, 1999.

(51) Int. Cl. C07K 14/475; C07H 21/04; C12N 1/21

(52) U.S. Cl. 435/69.1; 435/320.1; 435/252.3; 530/399; 536/23.5

(57) ABSTRACT

This invention concerns novel vascular endothelial growth factor (VEGF) dimers, compositions containing such dimers, processes for making such dimers, and methods for the treatment of various diseases by administering such dimers or compositions.

### BACKGROUND OF THE INVENTION

1. Field of the Invention

The present invention concerns novel vascular endothelial growth factor (VEGF) dimers, compositions containing such dimers, processes for making such dimers, and methods for the treatment of vascular diseases by administering such dimers and compositions.

2. Description of the Related Art

Vascular endothelial growth factor (VEGF), also referred to as vascular permeability factor (VPF), is a secreted protein generally occurring as a homodimer and having multiple biological functions. The native human VEGF monomer occurs as one of seven known isoforms, consisting of 121, 145, 148, 165, 183, 189, and 206 amino acid residues in length after removal of the signal peptide. These isoforms, either their monomeric or homodimeric form, are generally referred to in the literature as hVEGF₁₂₁, hVEGF₁₄₅, hVEGF₁₄₈, hVEGF₁₆₅, hVEGF₁₈₃, hVEGF₁₈₉, and hVEGF₂₀₆, respectively. The known isoforms are generated by alternative splicing of the RNA encoded by a single human VEGF gene that is organized in eight exons, separated by seven introns, and has been assigned to chromosome 6p21.3 (Vincenti et al., Circulation 93:1493-1495 [1996]). These isoforms are thus also referred to as VEGF splice variants. A schematic representation of the various forms of VEGF generated by alternative splicing of VEGF mRNA is shown in FIG. 2, where the protein sequences encoded by each of the eight exons of the VEGF gene are represented by numbered boxes. hVEGF₁₆₅ lacks the residues encoded by exon 6, while hVEGF₁₂₁ lacks the residues encoded by exons 6 and 7. hVEGF₁₂₁ is the only VEGF isoform known to be unable to bind to heparin. The lack of a heparin-binding region in hVEGF₁₂₁ has a profound effect on its biochemical and pharmacokinetic properties. In addition, proteolytic cleavage of hVEGF by plasmin produces a 110-amino acid proteolytic fragment species (hVEGF₁₁₀) (Keyt et al, J. Biol. Chem. 271:7788-7795 [1996]).

hVEGF₁₂₁ and hVEGF₁₆₅ are the most abundant of the seven known isoforms. hVEGF₁₂₁ and hVEGF₁₆₅ dimers both bind to the receptors KDR/Flk-1 and Flt-1 but hVEGF₁₆₅ dimers additionally bind to a more recently discovered receptor (VEGF₁₆₅R) (Soker et al., J. Biol. Chem. 271:5761-5767 [1996]). VEGF₁₆₅R has been recently cloned by Soker et al., and shown to be equivalent to a previously-defined protein known as neuropilin-1 (Cell 92:735-745 [1998]). The binding of hVEGF₁₆₅ dimer to the latter receptor is mediated by the exon-7 encoded domain, which is not present in hVEGF₁₂₁.

Dimeric VEGF is a potent mitogen for micro- and macrovascular endothelial cells derived from arteries, veins, and lymphatics, but shows significant mitogenic activity for virtually no other normal cell types. The denomination of VEGF reflects this narrow target cell specificity. As a result of its pivotal role in angiogenesis (spouting of new blood vessels) and vascular remodeling (enlargement of preexisting vessels), VEGF is a promising candidate for the treatment of coronary artery disease and peripheral vascular disease. High levels of VEGF are expressed in various types of tumors in response to tumor-induced hypoxia (Dvorak et al., J. Exp. Med. 174:1275-1278 [1991]; Plate et al., Nature 359:845-848 [1992]), and tumor growth has been inhibited by anti-VEGF antibodies and soluble VEGF receptors (Kim et al., Nature 362:841-844 [1993]; Kendall and Thomas, PNAS USA 90:10705-10709 [1993]).

The biologically active native form of hVEGF₁₂₁ is a homodimer (in which the two chains are in anti-parallel orientation) containing one N-linked glycosylation site per monomer chain at amino acid position 75 (Asn-75), which corresponds to a similar glycosylation site at position 75 of hVEGF₁₆₅. If the N-linked glycosylation structures are not present, the biologically active hVEGF₁₂₁ homodimer has a molecular weight of about 28 kDa with a calculated pI of 6.5. Each monomer chain in the hVEGF₁₂₁ homodimer has a total of nine cysteines, of which six are involved in the formation of three intrachain disulfides stabilizing the monomeric structure, and two are involved in two interchain disulfide bonds stabilizing the dimeric structure; until recently, one cysteine (Cys-116) has been believed to remain unpaired. Although Keck et al. (Arch. Biochem. Biophys. 344:103-113 [1997]) also identified an E. coli derived recombinant VEGF₁₂₁ dimer species having a Cys(116)-Cys(116) interchain disulfide bond, these authors stated that the unpaired cysteinie at position 116 of hVEGF₁₂₁ may nonetheless have biological significance, as it might, for example, serve to covalently anchor VEGF₁₂₁ to an extracellular matrix-associated protein, such a fibronectin, containing an unpaired cysteine (Wagner and Hynes, J. Biol. Chem. 254:6746-6754 [1979]).

hVEGF₁₂₁ has been expressed in E. coli (Keck et al., supra; Christinger et al., Prot. Struc. Func. Genet. 26:353-357 [1996]; Siemeister et al., Biochem. Biophys. Res. Comm. 222:249-255 [1996]; Siemeister et al., J. Biol. Chem. 273:11115-11120 [1998]; and Keyt et al., supra); by stable and transient expression in mammalian cell lines (Houck et al., J. Biol. Chem. 267:26031-26037 [1992]; Houck et al., Mol. Endo. 5:1806-1814 [1991]; and Siemeister et al., J. Biol. Chem., supra [1998]); in yeast, such as S. cerevisiae (Kondo et al., Biochim. Biophys. Acta 1243:195-202 [1995]), and P. pastoris (Mohanraj et al., Biochem. Biophys. Res. Comm. 215:750-756 [1995]); and in insect cells infected with a baculovirus-based expression system (Fiebich et al., Eur. J. Biochem. 211:19-26 [1993]; Cohen et al., J. Biol. Chem. 270:11322-11326 [1995]; and Gitay-Goren et al., J. Biol. Chem. 271:5519-5523 [1996]). Siemeister et al., J. Biol. Chem. supra (1998), have identified a domain between His-12 and Asp-19 in the amino acid sequence of hVEGF₁₂₁ as essential both for in vitro dimerization of recombinant VEGF₁₂₁ monomers, and for functional expression of this molecule in mammalian cells. There have been no reported studies concerning the potential effect of the state of Cys-116 in VEGF₁₂₁ on the biological activity, stability and other properties of this molecule.

### SUMMARY OF THE INVENTION

The present invention is based on the recognition that VEGF₁₂₁ dimers in which Cys-116 is disulfide bonded to another, extraneous cysteine have enhanced stability while retaining VEGF biological activity. The invention is further based on the finding that this is true not only for full-length (121 amino acids long) human VEGF₁₂₁, and its homologues in other animal, e.g. manmnalian species, but also for VEGF₁₂₁ derivatives, in particular variants that are variously truncated at the amino and/or carboxy terminus of the native VEGF₁₂₁ molecule, as long as in each of their monomer subunits, these variants retain a cysteine at a position corresponding to Cys-116 in the full-length human VEGF₁₂₁ molecule.

Accordingly, in one aspect, the invention concerns a vascular endothelial growth factor (VEGF) dimer consisting of a first and a second monomer each comprising at least amino acids 11 to 116 of SEQ ID NO: 1, or an amino acid sequence having at least about 90%, preferably at least about 95%, more preferably at least about 98% sequence identity with SEQ ID NO: 1, or with amino acids 11 to 116 of SEQ ID NO: 1, and retaining a cysteine at a position corresponding to position 116 of SEQ ID NO: 1 (Cys-116), wherein Cys-116 of each monomer is disulfide-bonded to an additional extraneous cysteine (Cys). The additional Cys may be part of a peptide comprising 2 to 5, preferably 2 to 3 amino acids, e.g. glutathione. Each monomer may be independently glycosylated or unglycosylated.

In another aspect, the invention concerns a composition comprising a VEGF dimer consisting of a first and a second monomer each comprising at least amino acids 11 to 116 of SEQ ID NO: 1, or an amino acid sequence having at least about 90%, preferably at least about 95%, more preferably at least about 98% sequence identity with SEQ ID NO: 1, or with amino acids 11 to 116 of SEQ ID NO: 1, and retaining a cysteine (Cys) at a position corresponding to position 116 of SEQ ID NO: 1 (Cys-116), wherein Cys-116 of each monomer is disulfide bonded to an additional Cys, in admixture with a pharmaceutically acceptable vehicle. Each monomer may be independently glycosylated or unglycosylated. In a preferred embodiment, the composition is essentially free of a VEGF dimer in which the cysteines at position 116 of each monomer are connected with an interchain disulfide bond and/or in which the cysteines at position 116 of each monomer are unpaired.

In yet another aspect, the invention concerns compositions of matter comprising at least two vascular endothelial growth factor (VEGF) dimers, each formed by a first and a second monomer, selected from the group consisting of:

(a) a dimer in which each monomer comprises amino acids 11 to 116 of SEQ ID NO: 1, or an amino acid sequence having at least about 90%, preferably at least about 95%, more preferably at least about 98% sequence identity with SEQ ID NO: 1, or with amino acids 11 to 116 of SEQ ID NO: 1, and retaining a cysteine (Cys) at a position corresponding to position 116 of SEQ ID NO: 1 (Cys-116), and the Cys at or corresponding to position 116 of each monomer is disulfide-bonded to an additional Cys;

(b) a dimer in which each monomer comprises amino acids 11 to 116 of SEQ ID NO: 1, or an amino acid sequence having at least about 90%, preferably at least about 95%, more preferably at least about 98% sequence identity with SEQ ID NO: 1, or with amino acids 11 to 116 of SEQ ID NO: 1, and retaining a cysteine (Cys) at a position corresponding to position 116 of SEQ ID NO: 1 (Cys-116), and the cysteines at or corresponding to position 116 of each monomer are connected with an interchain disulfide bond; and

(c) a dimer in which each monomer comprises amino acids 11 to 116 of SEQ ID NO: 1, or an amino acid sequence having at least about 90%, preferably at least about 95%, more preferably at least about 98% sequence identity with SEQ ID NO: I, or with amino acids 11 to 116 of SEQ ID NO: 1, and retaining a cysteine (Cys) at a position corresponding to position 116 of SEQ ID NO: 1 (Cys-116), and the Cys at or corresponding to position 116 of one or both monomers is unpaired;

wherein in each of said dimers (a)-(c) said first and second monomers may be independently glycosylated or unglycosylated. In a preferred embodiment, the composition comprises, as its main VEGF protein component, a dimer in which each monomer comprises amino acids 1 to 120 of SEQ ID NO: 1, or an amino acid sequence having at least about 90%, preferably at least about 95%, more preferably at least about 98% sequence identity with amino acids 1 to 120 of SEQ ID NO: 1 and retaining a cysteine at a position corresponding to position 116 of SEQ ID NO: 1 (Cys-116), and Cys-116 of each monomer is disulfide bonded to an additional Cys. This main component preferably constitutes at least about 60%, more preferably at least about 65%, more preferably at least about 70%, still more preferably at least about 75%, even more preferably at least about 80%, even more preferably at least about 85%, even more preferably at least about 90%, and most preferably at least about 95% of the amount of VEGF dimers present.

In a further aspect, the invention concerns a process for providing a composition of matter comprising VEGF polypeptides, wherein the VEGF polypeptides consist essentially of at least two vascular endothelial growth factor (VEGF) dimers, each formed by a first and a second monomer, selected from the group consisting of:

(a) a dimer in which each monomer comprises amino acids 11 to 116 of SEQ ID NO: 1, or an amino acid sequence having at least about 90%, preferably at least about 95%, more preferably at least about 98% sequence identity with SEQ ID NO: 1, or with amino acids 11 to 116 of SEQ ID NO: 1, and retaining a cysteine (Cys) at a position corresponding to position 116 of SEQ ID NO: 1 (Cys-116), and the Cys at or corresponding to position 116 of each monomer is disulfide-bonded to an additional Cys;

(b) a dimer in which each monomer comprises amino acids 11 to 116 of SEQ ID NO: 1, or an amino acid sequence having at least about 90%, preferably at least about 95%, more preferably at least about 98% sequence identity with SEQ ID NO: 1, or with amino acids 11 to 116 of SEQ ID NO: 1, and retaining a cysteine (Cys) at a position corresponding to position 116 of SEQ ID NO: 1 (Cys-116), and the cysteines at or corresponding to position 116 of each monomer are connected with an interchain disulfide bond; and

(c) a dimer in which each monomer comprises amino acids 11 to 116 of SEQ ID NO: 1, or an amino acid sequence having at least about 90%, preferably at least about 95%, more preferably at least about 98% sequence identity with SEQ ID NO: 1, or with amino acids 11 to 116 of SEQ ID NO: 1, and retaining a cysteine (Cys) at a position corresponding to position 116 of SEQ ID NO: 1 (Cys-116), and the Cys at or corresponding to position 116 of one or both monomers is unpaired;

wherein in each of dimers (a)-(c) the first and second monomers may be independently glycosylated or unglycosylated

The process comprises the steps of:

providing transformed host cells comprising a species of exogenously added DNA encoding a polypeptide of SEQ ID NO: 1, or encoding a polypeptide the amino acid sequence of which has at least about 90%, preferably at least about 95%, more preferably at least about 98% sequence identity with SEQ ID NO: 1, and retains a cysteine at a position corresponding to position 116 of SEQ ID NO: 1 (Cys-116), present in an operable expression vector,

culturing the host cells under conditions suitable for expression of said DNA and the synthesis of the VEGF polypeptides, and

recovering the VEGF polypeptides.

The process may comprise additional steps, including, for example, purification and/or refolding steps. When the transformed host cells are bacterial, e.g. E. coli cells, the polypeptides are typically refolded. In a preferred embodiment, the refolding buffer comprises cysteine and cystine in amounts and in a ratio relative to each other sufficient to produce the desired mixture of VEGF dimers.

If the host cells are bacterial cells, it is advantageous to use a DNA encoding a polypeptide of SEQ ID NO: 1 extended by a Met(AA)ₙ- sequence at the amino terminus (N-terminus), wherein Met stands for a methionine residue, n is 1-7, and AA represents identical or different amino acids, wherein at least one of the AA amino acids, or a combination of two or more AA amino acids, is capable of retardig proteolytic degradation of the mature N-terminus of the VEGF polypeptides in the bacterial cells. In a preferred embodiment, n stands for 1-5, preferably 1-3, more preferably 1 or 2, most preferably 1, and AA represents a lysine (Lys) or arginine (Arg) residue, preferably a Lys residue.

The invention further concerns a process for producing a vascular endothelial growth factor (VEGF) dimer composed of two VEGF monomers, in which each monomer comprises amino acids 11 to 116 of SEQ ID NO: 1, or comprises an amino acid sequence having at least about 90% sequence identity with amino acids 11 to 116 of SEQ ID NO: 1 and retaining a cysteine (Cys) at a position corresponding to position 116 of SEQ ID NO: 1 (Cys-116), where Cys-116 of each monomer is disulfide bonded to an additional extraneous Cys comprising the steps of:

(a) providing transformed bacterial host cells comprising a species of exogenously added DNA encoding a polypeptide of SEQ ID NO: 1 extended by a Met-(AA)ₙ- sequence at the amino terminus (N-terminus), wherein Met stands for methionine, n is 1-7, and AA represents identical of different amino acids, where at least one of the AA amino acids, or a combination of two or more AA amino acids, is capable of retarding proteolytic degradation of the mature N-terminus of the VEGF polypeptides formed by the bacterial host cells, present in an operable expression vector,

(b) culturing the bacterial host cells under conditions suitable for expression of said DNA and the synthesis of said VEGF monomers, and

(c) recovering the VEGF dimer.

Again, in a preferred embodiment, n stands for 1-5, preferably 1-3, more preferably 1 or 2, most preferably 1, and AA represents a lysine (Lys) or arginine (Arg) residue, preferably a Lys residue.

In a general aspect, the invention concerns a process for blocking the degradation of, e.g. removal of one or more amino acids from, the mature amino terminal (N-terminal) sequence of a polypeptide during production in a bacterial host cell by transforming the host cell with DNA encoding the polypeptide extended at its N-terminus by a Met-(AA)ₙ sequence, where Met stands for methionine, n is 1-7, and AA represents identical or different amino acids, where at least one of the AA amino acids, or a combination of two or more of the AA amino acids, is capable of retarding proteolytic degradation of the mature N-terminus of the polypeptide by the bacterial host cell. Just as before, n preferably is 1 to 5, more preferably 1 to 3, even more preferably 1 or 2, most preferably 1, and AA preferably stands for a lysine (Lys) or arginine (Arg) residue, preferably a Lys residue. The polypeptide preferably is longer than 100 amino acids, and preferably has at least about 120 amino acids. In a particularly preferred embodiment, the polypeptide is a native or variant VEGF polypeptide, more preferably, a native VEGF polypeptide, most preferably a hVEGF₁₂₁ or a hEGF₁₆₅ polypeptide.

In a still further aspect, the invention concerns methods of inducing angiogenesis or vascular remodeling, methods for the treatment of peripheral vascular disease, coronary artery disease, essential hypertension, microvascular angiopathy, and polycystic kidney disease, and methods for the repair of vascular endothelial cell layers, by administering the VEGF dimers or compositions of the present invention.

In all aspects of the invention, in a particularly preferred embodiment each VEGF monomer has an amino acid sequence consisting essentially of amino acids 1 to 121 of SEQ ID NO: 1, in which the glycosylation addition site at amino acid positions 75-77 may optionally be removed or altered such that glycosylation does not occur.

### BRIEF DESCRIPTION OF THE DRAWINGS

FIG. 1 shows the amino acid sequence and the encoding nucleotide sequence of native hVEGF₁₂₁, including the signal peptide. The signal peptide and the nucleotide sequence encoding the signal peptide are marked by underlining, and Cys-116 is marked with a double underline. SEQ ID NO: 1 shows the mature hVEGF₁₂₁ polypeptide (amino acids 1 to 121 in FIG. 1); SEQ ID NO: 2 shows the hVEGF₁₂₁ polypeptide including the signal peptide (amino acids -26 to -1 in FIG. 1); and SEQ ID NO: 3 shows the nuclotide sequence encoding the hVEGF₁₂₁ polypeptide including the signal peptide.

FIG. 2 is a schematic representation of the various forms of VEGF generated by alternative splicing of VEGF mRNA, where the protein sequences encoded by each of the eight exons of the VEGF gene are represented by numbered boxes. The protein sequences encoded by exon 1 and the first portion of exon 2 (shown as narrower boxes) represent the secretion signal sequence for VEGF.

FIG. 3 schematically illustrates the structure of a VEGF₁₂₁ dimer, in which Cys-116 is disulfide bonded to an “R” residue, where R is a cysteine, or a cysteine-containing peptide.

FIG. 4 schematically illustrates the structure of a VEGF₁₂₁ dimer, in which Cys-116 of each monomer participates in an interchain disulfide bond.

FIG. 5 schematically illustrates the structure of a VEGF₁₂₁ dimer, in which Cys-116 of each monomer is unpaired.

FIG. 6 illustrates the crystal structure of VEGF (8-109) dimer (Muller, et al., PNAS USA 94:7192-7197 [1997]). Intrachain disulfide bonds are shown between residues 104-61, 102-57 and 26-68 of the VEGF monomers, while interchain disulfide bonds are indicated between amino acid residues 51-60 and 60-51 of the two chains making up the VEGF dimer.

FIG. 7 shows the structure of an expression plasmid, used for the expression of hVEGF₁₂₁ in Chinese Hamster Ovary (CHO) cells, as described in Example 1.

FIG. 8 is a schematic diagram of E. coli expression plasmid pAN 179.

FIG. 9 is a schematic diagram of P. pastoris expression plasmid pAN103.

FIGS. 10 and 11 show the results of a comparative stability test of partially reduced VEGF₁₂₁ dimer (FIG. 10) and VEGF₁₂₁ dimer in which Cys-116 of each monomer is disulfide-bonded to an additional cysteine (FIG. 11), using reverse-phase HPLC chromatography.

FIG. 12 shows the results of a HWE cell proliferation assay (BrdU ELISA). The graph depicts the amount of DNA synthesis that was stimulated in response to serial dilutions of Pichia-derived N75Q VEGF₁₂₁ (VEGF standard; primarily consisting of molecules containing three interchain disulfide bonds) vs. E. coli-derived VEGF₁₂₁ (primarily consisting of molecules with only two interchain disulfide bonds, with additional extraneous cysteines disulfide-bonded to the Cys-116 residues). The X axis of the graph represents the final concentration of added growth factor in the assay wells, expressed as ng/ml. The Y axis represents the optical density recorded in each well after use of the BrdU kit (Boehringer Mannheim) to detect incorporated bromodeoxyuridine (BrdU) at the end of the assay.

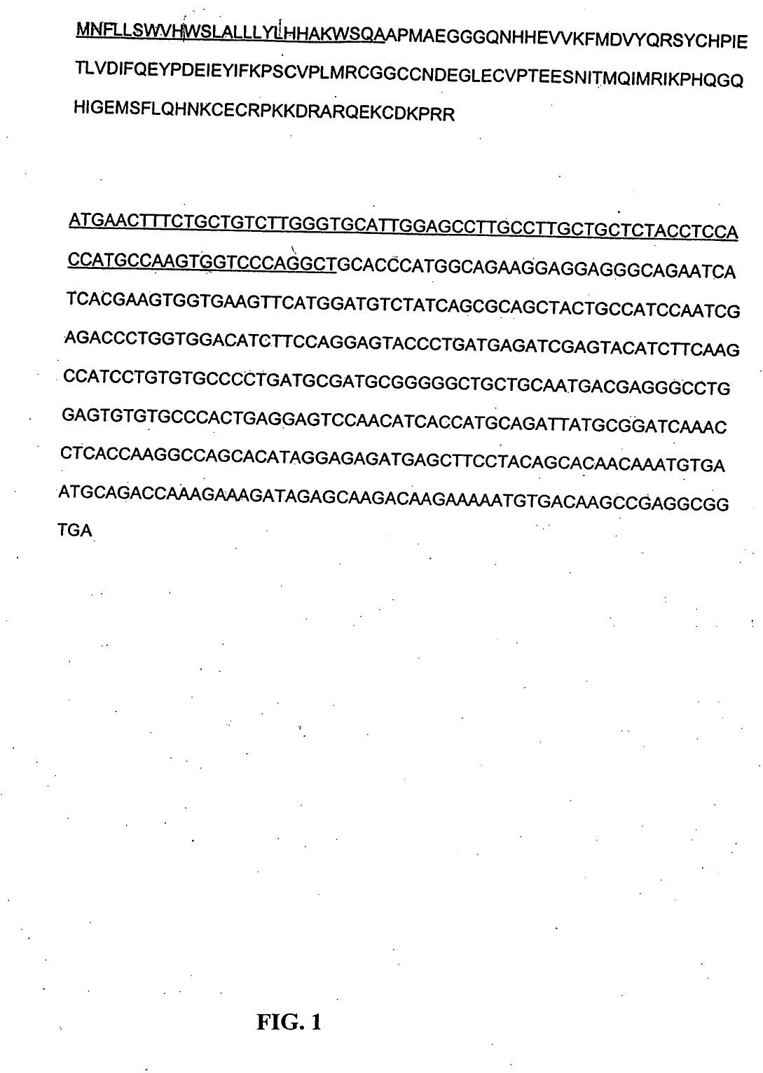

### DETAILED DESCRIPTION OF THE INVENTION

The practice of the present invention will employ, unless otherwise indicated, conventional, techniques of molecular biology (including recombinant techniques), microbiology, cell biology, biochemistry and immunology, which are within the skill of the art. Such techniques are explained fully in the literature, such as, “Molecular Cloning: A Laboratory Manual”, second edition (Sambrook et al., 1989); “Oligonucleotide Synthesis” (Gait, ed., 1984); “Animal Cell Culture” (Freshney, ed., 1987); “Methods in Enzymology” (Academic Press, Inc.); “Handbook of Experimental Immunology” (Weir & Blackwell, eds.); “Gene Transfer Vectors for Mammalian Cells” (Miller & Calos, eds., 1987); “Current Protocols in Molecular Biology” (Ausubel et al., eds., 1987); “PCR: The Polymerase Chain Reaction” (Mullis et al., eds., 1994); and “Current Protocols in Immunology” (Coligan et al., eds., 1991).

Definitions

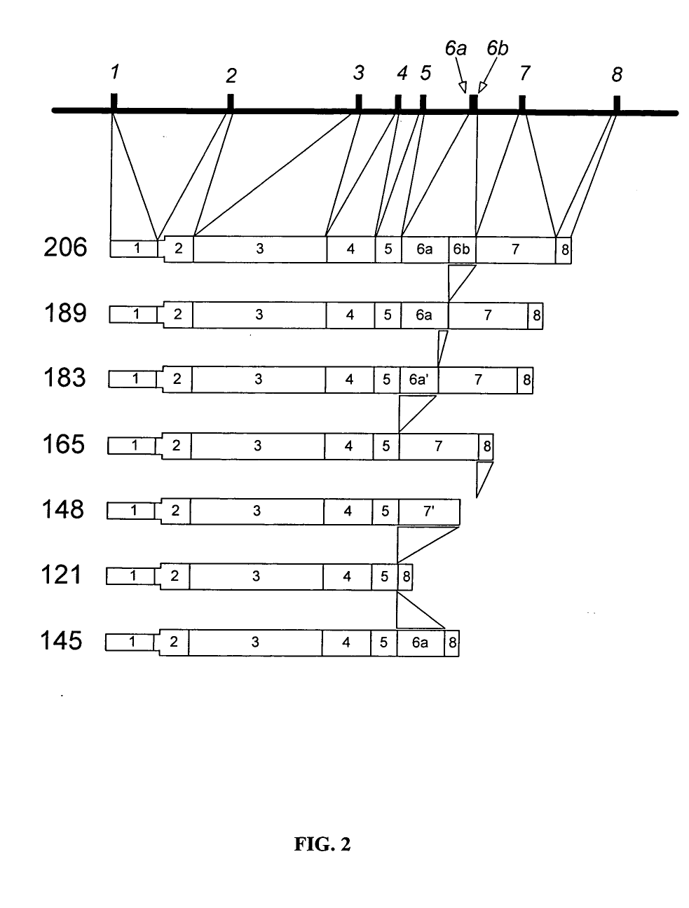

The term “vascular endothelial growth factor” or “VEGF” as used herein refers to any naturally occurring (native) forms of a VEGF polypeptide (also known as “vascular permeability factor” or “VPF”) from any animal species, including humans and other mammalian species, such as murine, rat, bovine, equine, porcine, ovine, canine, or feline, and functional derivatives thereof, in monomeric or dimeric form. “Native human VEGF” consists of two polypeptide chains, and generally represents a homodimer, and will be generally referred to as “native human VEGF dimer”. Each monomer occurs as one of seven known isoforms, consisting of 121, 145, 148, 165, 183, 189, and 206 amino acid residues in length. These isoforms will be hereinafter referred to as hVEGF₁₂₁, hVEGF₁₄₅, hVEGF₁₄₁, hVEGF₁₆₅, hVEGF₁₈₃, hVEGF₁₈₉, and hVEGF₂₀₆, respectively, again, including their monomeric and homodimeric forms. Similarly to the human VEGF, “native murine VEGF”, “native rat VEGF” and “native ovine VEGF” are also known to exist in several isoforms, 120, 164, and 188 amino acids in length, usually occurring as homodimers. In addition, “native bovine VEGF” is known to exist in at least two isoforms, 120 and 164 amino acids in length, usually occurring as homodimers. With the exception of hVEGF₁₂₁ dimer, all native human VEGF dimers are known or believed to be basic, heparin-binding molecules. hVEGF₁₂₁ dimer is a weakly acidic protein that does not bind to heparin. These and similar native forms, whether known or hereinafter discovered are all included in the definition of “native VEGF” or “native sequence VEGF”, regardless of their mode of preparation, whether isolated from nature, synthesized, produced by methods of recombinant DNA technology, or any combination of these and other techniques. The term “vascular endothelial growth factor” or “VEGF” includes VEGF polypeptides in monomeric, homodimeric and heterodimeric forms. The definition of “VEGF” also includes a 110 amino acids long human VEGF proteolytic fragment species (hVEGF₁₁₀), and its homologues in other mammalian species, such as murine, rat, bovine, equine, porcine, ovine, canine, or feline, and functional derivatives thereof. In addition, the term “VEGF” covers chimeric, dimeric proteins, in which a portion of the primary amino acid structure corresponds to a portion of either the A-chain subunit or the B-chain subunit of platelet-derived growth factor, and a portion of the primary amino acid structure corresponds to a portion of a native or variant vascular endothelial growth factor. In a particular embodiment, a chimeric molecule is provided consisting of one chain comprising at least a portion of the A- or B-chain subunit of a platelet-derived growth factor, disulfide linked to a second chain comprising at least a portion of a native or variant VEGF molecule, such as VEGF₁₂₁. More details of such dimers are provided, for example, in U.S. Pat. Nos. 5,194,596 and 5,219,739 and in European Patent EP-B 0 484 401, the disclosures of which are hereby expressly incorporated by reference. The nucleotide and amino acid sequences of hVEGF₁₂₁ and bovine VEGF₁₂₀ are disclosed, for example, in U.S. Pat. Nos. 5,194,596 and 5,219,739, and in EP-B 0 484 401. hVEGF₁₄₅ is described in U.S. Pat. No. 6,013,780 and PCT Publication No. WO 98/10071; hVEGF₁₆₅ is described in U.S. Pat. No. 5,332,671; hVEGF₁₈₉ is described in U.S. Pat. No. 5,240,848; and hVEGF₂₀₆ is described in Houck et al. Mol. Endo. supra (1991). For the disclosure of the nucleotide and amino acid sequences of various human VEGF isoforms see also Leung et al., Science 246:1306-1309 (1989); Keck et al., Science 246:1309-1312 (1989); Tischer et al., J. Biol. Chem. 266:11947-11954 (1991); EP 0 370 989; and PCT publication WO 98/10071. Forms of VEGF are shown schematically in FIG. 2.

“Human VEGF₁₂₁ monomer” or “hVEGF₁₂₁ monomer” is defined herein as a polypeptide of SEQ ID NO: 1 (native or wild-type hVEGF₁₂₁ monomer), or a functional derivative thereof. Monomers of non-human homologues of hVEGF₁₂₁ (“VEGF₁₂₁ monomers” or “VEGF₁₂₀ monomers”) are defined in an analogous fashion.

“Human VEGF₁₂₁ dimer” or “hVEGF₁₂₁ dimer” is defined herein as a dimer of two identical hVEGF₁₂₁ monomers as hereinabove defined (“homodimer”), or a dimer formed between a hVEGF₁₂₁ monomer as hereinabove defined and another subunit (“heterodimer”) which differs in at least one aspect. For example, the two subunits (monomers) in a heterodimeric hVEGF₁₂₁ molecule may differ in the presence or absence of glycosylation. Thus, homodimers may have both of their subunits unglycosylated or glycosylated, while in heterodimers, one subunit may be glycosylated and the other unglycosylated. Similarly, the state of the Cys-116 residue, or a corresponding residue in a functional derivative of human VEGF₁₂₁, or a non-human VEGF₁₂₁ homologue may differ in the two monomeric chains of a heterodimer. Accordingly, the term “hVEGF₁₂₁ heterodimer” specifically includes not only dimers consisting of two monomers which differ in their amino acid sequence but also dimers consisting of two monomers which differ in their state or pattern of glycosylation, or state of the Cys-116 residue. “hVEGF₁₂₁ dimers” specifically cover chimeric, dimeric proteins, in which a portion of the primary amino acid structure corresponds to a portion of either the A-chain subunit or the B-chain subunit of platelet-derived growth factor, and a portion of the primary amino acid structure corresponds to a portion of VEGF₁₂₁. In a particular embodiment, a chimeric molecule is provided consisting of one chain comprising at least a portion of the A- or B-chain subunit of a platelet-derived growth factor, disulfide linked to a second chain comprising at least a portion of a hVEGF₁₂₁ molecule. More details of such dimers are provided, for example, in U.S. Pat. Nos. 5,194,596 and 5,219,739 and in European Patent EP-B 0 484 401. Dimers of non-human homologues of hVEGF₁₂₁ are defined in an analogous fashion.

The terms “human VEGF₁₂₁”, “hVEGF₁₂₁”, “native human VEGF₁₂₁” and “native hVEGF₁₂₁”, unless otherwise mentioned, include both hVEGF₁₂₁ monomers and hVEGF₁₂₁ dimers (including homo- and heterodimers), as hereinabove defined.

“VEGF₁₂₁” as used herein refers to native human VEGF₁₂₁ as hereinabove defined, its homologues in other non-human animals, e.g. Non-human mammalian species, and functional derivatives thereof. Again, unless otherwise mentioned, the term includes both VEGF₁₂₁ monomers and VEGF₁₂₁ dimers.

The amino acid sequence numbering system used herein for VEGF is based on the mature forms of the protein, i.e. the post-translationally processed forms. Accordingly, the residue numbered one in the human proteins is alanine, which is the first residue of the isolated, mature forms of these proteins (Connolly et al., J. Biol. Chem. 264:20017-20024 [1989]).

A “functional derivative” of a protein is a compound having a qualitative biological activity in common with the reference, e.g. native protein. A functional derivative of a VEGF₁₂₁ is a monomeric or dimeric VEGF molecule that retains at least one biological activity of a native VEGF₁₂₁, lacks heparin binding, and, in at least one VEGF monomer, has a cysteine at a position corresponding to amino acid position 116 of the native human VEGF₁₂₁ molecule. In addition, a “functional derivative” of a VEGF monomer includes derivatives of the monomer that can be incorporated into dimeric structures to create functional dimers, i.e., homodimers or heterodimers that retain at least one biological activity of a native VEGF molecule. “Functional derivatives” include, but are not limited to fragments of native polypeptides from any animal species (including humans), and derivatives of native (human and non-human) polypeptides and their fragments.

The terms “biological activity” and “activity” in connection with the VEGF₁₂₁ dimers of the present invention include mitogenic activity as determined in any in vitro assay of endothelial cell proliferation. This activity is preferably determined in a human umbilical vein endothelial (HUVE) cell-based assay, as described, for example, in any of the following publications: Gospodarowicz et al., PNAS USA 86:7311-7315 (1989); Ferrara and Henzel, Biochem. Biophys. Res. Commun. 161:851-858 (1989); Conn et al., PNAS USA 87:1323-1327 (1990); Soker et al, Cell, supra (1998); Waltenberger et al., J. Biol. Chem. 269:26988-26995 (1994); Siemeister et al., Biochem. Biophys. Res. Commun. supra (1996); Fiebich et al., supra; Cohen et al., Growth Factors 7:131-138 (1993). A further biological activity is involvement in angiogenesis and/or vascular remodeling, which can be tested, for example in the rat corneal pocket angiogenesis assay as described in Connolly et al., J. Clin. Invest. 84: 1470-1478 (1989); the endothelial cell tube formation assay, as described for example in Pepper et al., Biochem. Biophys. Res. Commun. 189:824-831 (1992), Goto et al., Lab. Invest. 69:508-517 (1993), or Koolwijk et al., J. Cell Biol. 132: 1177-1188 (1996); or the chick chorioallantoic membrane (CAM) angiogenesis assay as described for example in Plouet et al., EMBO J. 8: 3801-3806 (1989). Other preferred biological activities include, without limitation, enhancement of vascular permeability as determined in the Miles Assay (Connolly et al., J. Biol Chem. supra [1989]); and hypotensive activity, as determined in the hypotension assay described in Yang et al., J. Pharmacol. Experimental Therapeutics 284: 103-110 (1998).

“Fragments” comprise regions within the sequence of a mature native human VEGF₁₂₁, or a homologue in a non-human animal, e.g. non-human mammalian species.

The term “derivative” is used to define amino acid sequence and glycosylation variants, fragments, and covalent modifications of a native polypeptide, while the term “variant” refers to amino acid sequence and glycosylation variants within this definition.

The “amino acid sequence variants” are polypeptides (including dimers of polypeptides) in which one or more amino acids are added and/or substituted and/or deleted and/or inserted at the N- or C-terminus or anywhere within the corresponding native sequence, and which retain at least one activity of the corresponding native protein. In various embodiments, a “variant” polypeptide usually has at least about 75% amino acid sequence identity, or at least about 80% amino acid sequence identity, preferably at least about 85% amino acid sequence identity, even more preferably at least about 90% amino acid sequence identity, and most preferably at least about 95% amino acid sequence identity with the amino acid sequence of the corresponding native sequence polypeptide.

“Sequence identity” is defined as the percentage of amino acid residues in a candidate sequence that are identical with the amino acid residues at corresponding positions in a native polypeptide sequence, after aligning the sequences and introducing gaps if necessary, to achieve the maximum percent sequence identity, and not considering any conservative substitutions as part of the sequence identity. The % sequence identity values are generated by the NCBI BLAST2.0 software as defined by Altschul et al., “Gapped BLAST and PSI-BLAST: a new generation of protein database programs”, Nucleic Acids Res., 25:3389-3402 (1997). The parameters are set to default values, with the exception of Penalty for mismatch, which is set to −1.

The terms “extraneous cysteine” or “additional cysteine” or “additional extraneous cysteine” in the context of the present invention are used to refer to a cysteine that is not directly encoded by a nucleic acid sequence encoding the hVEGF₁₂₁ of SEQ ID NO: 1, its functional derivatives, or its homologues in another animal, e.g. non-human mammalian species. The structure in which, in at least one VEGF monomer, the cysteine at a position corresponding to position 116 in the native human VEGF₁₂₁ molecule is disulfide-bonded to an extraneous cysteine will also be referred to as a “mixed disulfide” structure. In some instances, the extraneous cysteine may be part of a peptide, such as a glutathione molecule.

The term “unpaired” in reference to a cysteine at a position corresponding to position 116 in the native human VEGF₁₂₁ molecule, designates a cysteine comprising a free sulfhydryl group.

The term “vector” is used herein in the broadest sense, and includes, but is not limited to, RNA, DNA, DNA encapsulated in an adenovirus coat, DNA packaged in another viral or viral-like form (such as herpes simplex, and adeno-associated virus (AAV)), DNA encapsulated in liposomes, and DNA complexed with polylysine, complexed with synthetic polycationic molecules, conjugated with transferrin, complexed with compounds such as polyethylene glycol (PEG) to immunologically “mask” the molecule and/or increase half-life, or conjugated to a non-viral protein. Preferably, the vector is a DNA vector.

As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A “host cell” includes an individual cell or cell culture which can be or has been a recipient of any vector of this invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo with a vector comprising a polynucleotide encoding a VEGF.

An “individual” is a vertebrate, preferably a mammal, more preferably a human. Mammals include, but are not limited to, farm animals, sport animals, and pets.

An “effective amount” is an amount sufficient to effect beneficial or desired clinical results. An effective amount can be administered in one or more administrations. For purposes of this invention, an effective amount of a VEGF dimer or composition is an amount that is sufficient to palliate, ameliorate, stabilize, reverse, slow or delay the progression of the targeted disease state.

“Mammal” for purposes of treatment refers to any animal classified as a mammal, including humans, domestic and farm animals, and zoo, sports, or pet animals, such as horses, sheep, cows, pigs, dogs, cats, etc. Preferably, the mammal is human.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, sucrose, mannose, trehalose, or dextrins; chelating agents such as ethylenediaminotetraacetic acid (EDTA); sugar alcohols such as mannitol or sorbitol; salt-forming counterions such as sodium; and/or nonionic surfactants such as TWEEN®, polyethylene glycol (PEG), and PLURONICS®.

“Angiogenesis” is defined as the promotion of the growth of new capillary blood vessels from existing vasculature, while “therapeutic angiogenesis” is defined as the promotion of the growth of new blood vessels and/or remodeling of existing blood vessels, for example, to increase blood supply to an ischemic region.

The term “peripheral arterial disease” also known as “peripheral vascular disease”, is defined as the narrowing or obstruction of the blood vessels supplying the extremities. It is a common manifestation of atherosclerosis, and most often affects the blood vessels of the leg. Two major types of peripheral arterial disease are intermittent claudication, in which the blood supply to one or more limbs has been reduced to the point where exercise cannot be sustained without the rapid development of cramping pain; and critical leg ischemia, in which the blood supply is no longer sufficient to completely support the metabolic needs of even the resting limb.

“Coronary artery disease” is defined as the narrowing or obstruction of one or more arteries that supply blood to the muscle tissue of the heart. This disease is also a common manifestation of atherosclerosis.

The term “microvascular angiopathy” is used to describe acute injuries to smaller blood vessels and subsequent dysfunction of the tissue in which the injured blood vessels are located. Microvascular angiopathies are a common feature of the pathology of a variety of diseases of various organs, such as kidney, heart, and lungs. The injury is often associated with endothelial cell injury or death and the presence of products of coagulation or thrombosis. The agent of injury may, for example, be a toxin, an immune factor, an infectious agent, a metabolic or physiological stress, or a component of the humoral or cellular immune system, or may be as of yet unidentified. A subgroup of such diseases is unified by the presence of thrombotic microangiopathies (TMA), and is characterized clinically by non-immune hemolytic anemia, thrombocytopenia, and/or renal failure. The most common cause of TMA is the hemolytic uremic syndrome (HUS), a disease that is particularly frequent in childhood, where it is the most common cause of acute renal failure. The majority of these cases are associated with enteric infection with the verotoxin-producing strain, E. coli O157. Some HUS patients, especially adults, may have a relative lack of renal involvement and are sometimes classified as having thrombotic thrombocytopenic purpura (TTP). However, TMA may also occur as a complication of pregnancy (eclampsia), with malignant hypertension following radiation to the kidney, after transplantation (often secondary to cyclosporine or FK506 treatment), with cancer chemotherapies (especially mitomycin C), with certain infections (e.g., Shigella or HIV), in association with systemic lupus or the antiphospholipid syndrome, or may be idiopathic or familial. Experimental data suggest that endothelial cell injury is a common feature in the pathogenesis of HUS/TTP.

“Chronic” administration refers to administration of the agent(s) in a continuous mode as opposed to an acute mode, so as to maintain the initial therapeutic effect (activity) for an extended period of time.

“Intermittent” administration is treatment that is not consecutively done without interruption, but rather is cyclic in nature.

The term “essentially free” is used to mean that the undesired component (the component of which a composition is essentially free) represents less than about 2%, preferably less than about 1%, more preferably less than about 0.5%, even more preferably less than about 0.1%, most preferably less than about 0.05% of the composition.

The term “capable of retarding proteolytic degradation of the mature N-terminus” and grammatical equivalents thereof are used to describe the ability of amino acid(s), when added to a primary translation product (precursor) for a polypeptide, e.g. a VEGF polypeptide, between the initiating (N-terminal) methionine (Met) and the mature N-terminus of the polypeptide, to retard amino-terminal truncation of the desired mature polypeptide by proteases in the recombinant host cell. The extension delays or blocks the complete maturation of the amino terminus of the polypeptide product so that the polypeptide and/or its precursor forms can be removed from the host cell and purified away from protease(s) present in the host cell that, in the absence of the extension, would over time cleave residues representing the N-terminal end of the mature polypeptide. The extension is selected such that even if the initiating Met is removed from part of the product during fermentation, thereby exposing the remaining amino acids within the extension to proteolytic cleavage, the resultant N-terminal truncation of the precursor leaves intact the mature N-terminus of the polypeptide. The added N-terminal extension (Met-AAₙ), including the initiating Met, or the remainder of the extension, can then be removed in a controlled, purified enzymatic reaction as part of the recovery of the VEGF protein.

### DETAILED DESCRIPTION OF PREFERRED EMBODIMENTS

Native human VEGF₁₂₁ (hVEGF₁₂₁) is a VEGF isoform that differs from the other isoforms of the native human VEGF protein in a number of significant ways. All native human isoforms of VEGF, as defined herein, have a common amino terminal domain from residues 1 to 114, encoded by exons 2 through 5. However, hVEGF₁₂₁ contains in addition a lysine residue (encoded by the codon spanning the splice junction at the end of exon 5) and then only up to six more amino acids [CDKPRR] encoded by the carboxy terminal exon 8, and thus lacks the heparin-binding domains encoded by exons 6 and 7. Accordingly, hVEGF₁₂₁ is the only human VEGF isoform known not to bind to heparin. Furthermore, although hVEGF₁₂₁ dimers and hVEGF₁₆₅ dimers both bind to the receptors KDR/Flk-1 and Flt-1, hVEGF₁₆₅ dimers additionally bind to a more recently discovered receptor (VEGF₁₆₅R) (Soker et al., J. Biol. Chem. supra [1996]). Since the binding of hVEGF₁₆₅ to the latter receptor is mediated by the exon-7 encoded domain, which is not present in hVEGF₁₂₁, hVEGF₁₂₁ dimers do not bind VEGF₁₆₅R. A further significant difference between hVEGF₁₂₁ and the longer VEGF isoforms is in the disulfide structure of these molecules. The biologically active forms of all native VEGF molecules are disulfide-bonded dimers, primarily homodimers. The predominant larger form of native hVEGF, hVEGF₁₆₅, has a total of 16 cysteines in each monomer; in dimers of this isoform, two of the cysteines are involved in two interchain disulfide bonds, while the rest of the cysteines are involved in intrachain disulfide bonds. Each monomer chain in the hVEGF₁₂₁ homodimer has a total-of nine cysteines, of which six are involved in the formation of three intrachain disulfides stabilizing the monomeric structure, two are involved in two interchain disulfide bonds stabilizing the dimeric structure, while one cysteine (Cys-116) has been described as being unpaired.

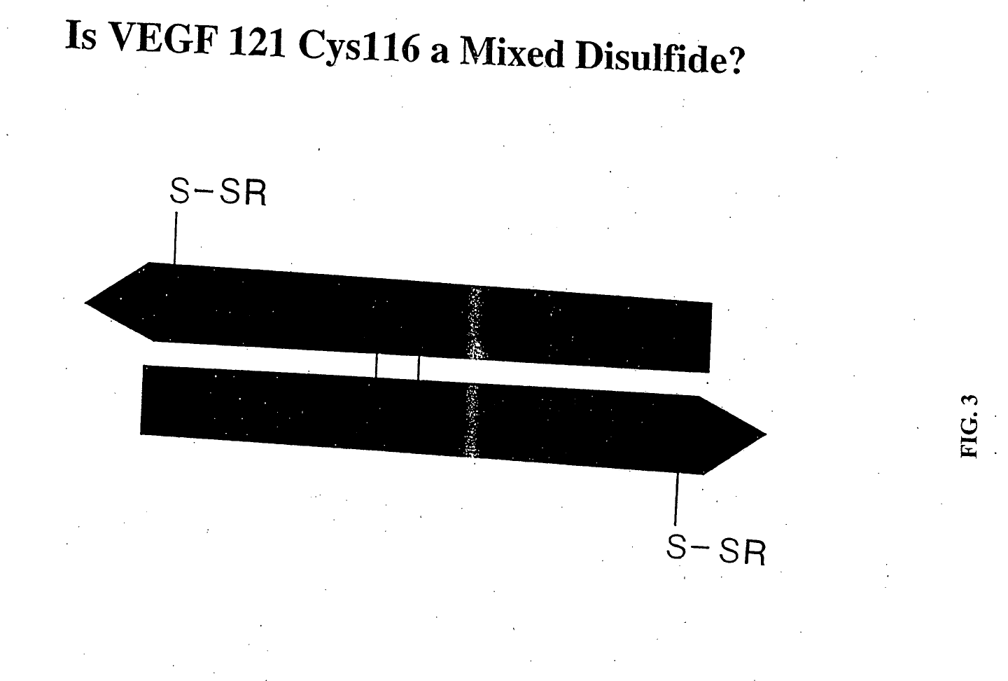

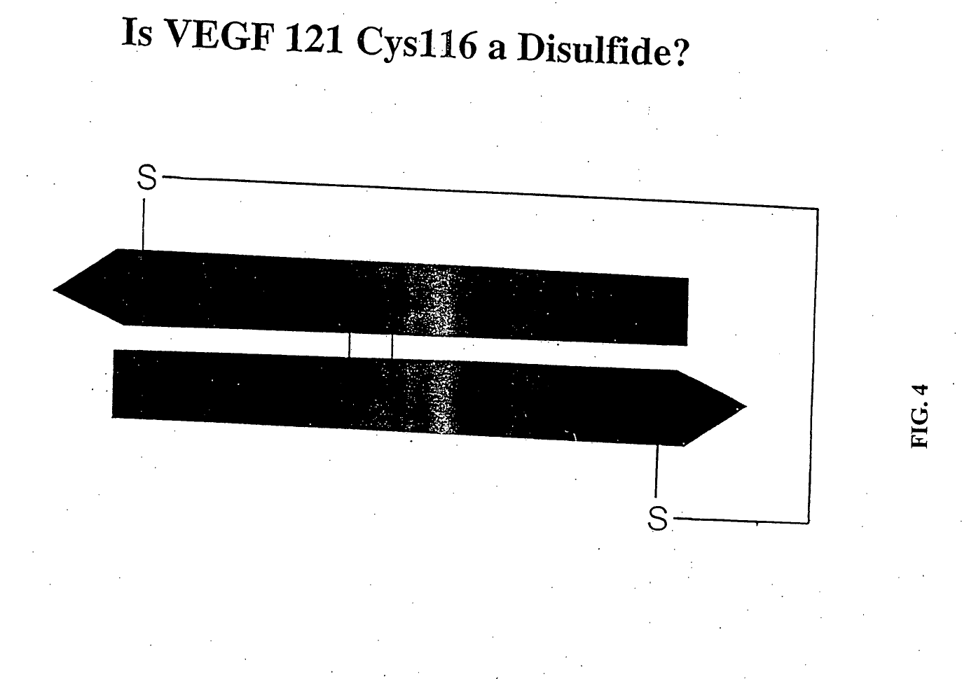

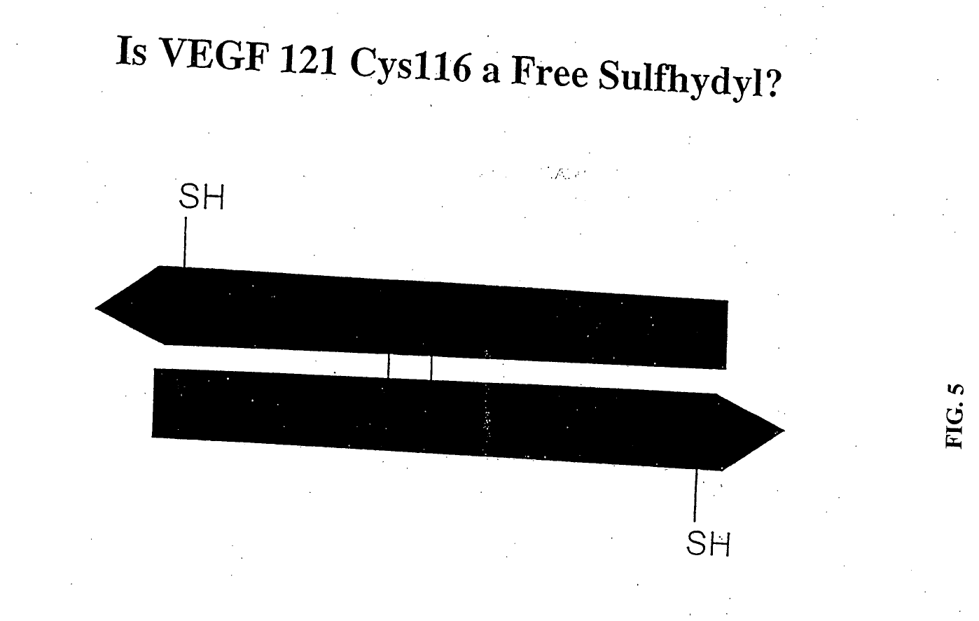

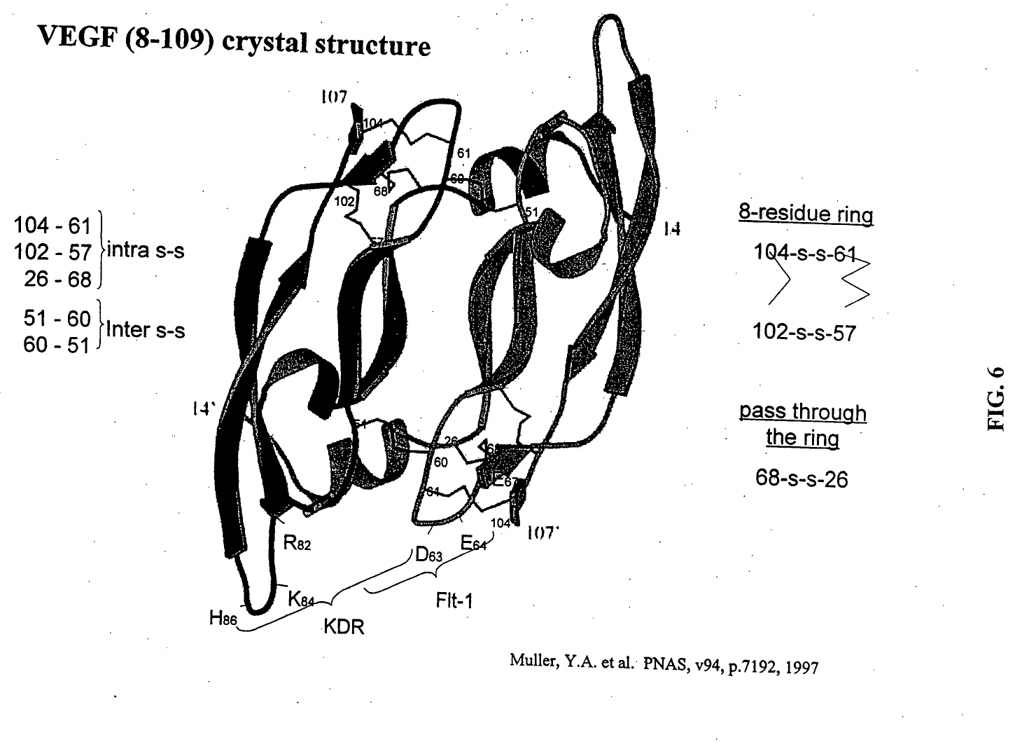

We have found that the state of Cys-116 has a profound effect on the stability of the hVEGF₁₂₁ molecule. Cys-116 can be disulfide bonded to an extraneous “R” moiety as shown in FIG. 3, where R is a cysteine or a cysteine-containing peptide, to form a “mixed disulfide” structure, or can participate in an interchain disulfide bond (FIG. 4), or can remain “unpaired” (FIG. 5). We have determined that by producing hVEGF₁₂₁ dimers in a form which contains a “mixed disulfide” at Cys-116 of at least one (preferably both) of the monomers, the stability of the hVEGF₁₂₁ dimer can be significantly enhanced, without compromising its biological activity, relative to the form of the dimer in which the cysteines at position 116 are “unpaired”. This is particularly surprising in view of earlier suggestions that the presence of an unpaired cysteine at position 116 may have biological significance (Keck et al., Arch. Biochem. Biophys. supra [1997]). Accordingly, the objective of the present invention is to produce, by means of recombinant DNA technology, hVEGF₁₂₁ dimers in which at least one, and preferably both, cysteines at positions 116 of the monomers, are disulfide-bonded to an extraneous cysteine.

We have additionally found that the stability and biological activity of recombinant hVEGF₁₂₁ dimers are not compromised by amino acid deletions, substitutions or insertions at the amino and/or carboxy terminus of the hVEGF₁₂₁ molecule.

We have specifically found that recombinant production of human VEGF₁₂₁ in mammalian cells, essentially following the procedure illustrated in the examples, yields a mixture of VEGF species, including variants having one or more amino acids deleted at the carboxy- and/or amino-terminus of the native human VEGF₁₂₁ molecule. For example, expression in Chinese hamster ovary (CHO) cells typically yields a mixture of a main species of 120 amino acids, having a correct amino terminus but missing the last amino acid of wild-type human VEGF₁₂₁, and some minor species, including variously truncated variants having up to 10 of their N-terminal amino acids deleted, and a 121 amino acids species. Typically, the 120 amino acids long VEGF species constitutes at least about 60%, preferably at least about 65%, more preferably at least about 70%, even more preferably at least about 75%, still more preferably at least about 80%, even more preferably at least about 85 %, more preferably at least about 90%, and most preferably at least about 95% of the final product. Expression in mammalian cells may be performed to produce a dimer in which Cys-116 in each monomer is predominantly attached to an extraneous cysteine via a disulfide bond. In a smaller fraction of the dimers produced, cysteines-116 in the two monomers are coupled by an interchain disulfide bond. In a particular embodiment, the expression is performed in the presence of glutathione. As a result, one or both cysteines at position 116 in the monomer subunits of the hVEGF₁₂₁ dimers may be disulfide bonded to a glutathione (γGlu-Cys-Gly) molecule. In addition to glutathione, other sulfhydryl-containing compounds can be disulfide-bonded to Cys-116. Such compounds include, without limitation, cystamine and coenzyme A. The carboxy and amino terminal truncations are believed to have no detrimental effect on the biological activity of the molecule.

We have further found that recombinant production of hVEGF₁₂₁ in yeast, following a procedure similar to that illustrated in the example, also produces a product mixture. For example, expression in Pichia pastoris (P. pastoris) yields, as a main component, a species truncated by four amino terminal and one carboxy terminal residues compared to the full-length native sequence. Accordingly, the predominant product in P. pastoris is composed of amino acids 5-120 of the native, full-length hVEGF₁₂₁ molecule. Small amounts (0.1-0.6%) of species initiating at residues 6, 7, 8, 11, 12 and 18 are also sometimes detected. The product is also a mixture of VEGF species, in which the cysteines at amino acid positions 116 of the two VEGF monomers are attached to extraneous cysteines (optionally present as part of a peptide, e.g. glutathione), or participate in the formation of a third interchain disulfide bond. Additionally, the mixture of VEGF species produced in P. pastoris can be converted into a much less complex mixture, in which the predominant form contains a mixed disulfide at position 116 of each monomer subunit, by (1) selectively reducing the cysteines at position 116, as described in the examples, and (2) allowing the resulting material to react with free cysteine, cystine, or Cys-containing peptide.

We have also found that recombinant production of hVEGF₁₂₁ in E. coli essentially as described in the examples, yields a product mixture comprising the full-length form as the main component. The mature full-length form usually makes up at least about 85%, preferably at least about 90%, more preferably at least about 95%, and even more preferably at least about 98% of the end product. The product may also contain some (typically about 1-2%) longer VEGF species, having extraneous amino acids at the N-terminus, and/or some (typically about 1-3%) shorter forms, missing up to four, such as one or four N-terminal amino acids. The E. coli-derived dimeric product will typically have a “mixed disulfide” structure at amino acid position 116, while, in a smaller percentage of the product obtained, the two cysteines-116 are connected to form a third interchain disulfide bond. The manufacturing process is preferably designed to minimize the presence of free (unpaired) sulfhydryl at position 116, and produce at least about 90% mixed disulfide, in which Cys-116 in each monomer is disulfide-bonded to an extraneous cysteine, which may be part of a peptide molecule, e.g. glutathione.

Typically, the cDNA encoding the monomeric chains of the desired VEGF₁₂₁ dimer is inserted into a replicable expression vector for cloning and expression. Suitable vectors are prepared by standard techniques of recombinant DNA technology, and are, for example, described in the textbooks cited above. Isolated plasmids and DNA fragments are cleaved, tailored, and ligated together in a specific order to generate the desired vectors. After ligation, the vector containing the gene to be expressed is transformed into a suitable host cell.

As noted before, host cells used for the production of the VEGF₁₂₁ dimers of the present invention can be any eukaryotic or prokaryotic hosts known for expression of heterologous proteins. Thus, the VEGF₁₂₁ dimers of the present invention can be expressed in eukaryotic hosts, such as eukaryotic microbes (yeast), or cells isolated from multicellular organisms (mammalian cell cultures, plant cells, and insect cell cultures), or in prokaryotic hosts, such as bacteria, e.g. E. coli.

Suitable yeast hosts include Saccharomyces cerevisiae (common baker's yeast), which is the most commonly used among lower eukaryotic hosts. However, a number of other genera, species, and strains are also available and useful herein, including Pichia pastoris. The expression of the VEGF₁₂₁ dimers of this invention in Pichia pastoris is specifically illustrated in the examples below. Other yeasts suitable for VEGF expression include, without limitation, Kluyveromyces hosts (U.S. Pat. No. 4,943,529), e.g. Kluyveromyces lactis; Schizosaccharomyces pombe (Beach and Nurse, Nature 290:140 (1981); Aspergillus hosts, e.g. A. niger (Kelly and Hynes, EMBO J. 4:475-479 [1985]) and A. nidulans (Ballance et al., Biochem. Biophys. Res. Commun. 112:284-289 [1983]), and Hansenula hosts, e.g. Hansenula polymorpha.

Preferably a methylotrophic yeast is used as a host in producing the VEGF₁₂₁ dimers of the present invention. Suitable methylotrophic yeasts include, but are not limited to, yeast capable of growth on methanol selected from the group consisting of the genera Pichia and Hansenula. A list of specific species which are exemplary of this class of yeasts may be found, for example, in C. Anthony, The Biochemistry of Methylotrophs, 269 (1982). Presently preferred are methylotrophic yeasts of the genus Pichia such as the auxotrophic Pichia pastoris GS115 (NRRL Y-15851); Pichia pastoris GS190 (NRRL Y-18014) disclosed in U.S. Pat. No. 4,818,700; and Pichia pastoris PPF1 (NRRL Y-18017) disclosed in U.S. Pat. No. 4,812,405. Auxotrophic Pichia pastoris strains are also advantageous to the practice of this invention for the ease of selecting transformed progeny containing VEGF₁₂₁ expression vectors. It is recognized that wild type Pichia pastoris strains (such as NRRL Y-11430 and NRRL Y-11431) may be employed with equal success if a suitable transforming marker gene is selected, such as the use of SUC2 to transform Pichia pastoris to a strain capable of growth on sucrose, or if an antibiotic resistance marker is employed, such as resistance to G418. Pichia pastoris linear plasmids are disclosed, for example, in U.S. Pat. No. 5,665,600.

Suitable promoters used in yeast vectors include the promoters for 3-phosphoglycerate kinase (Hitzeman et al., J. Biol. Chem. 255:2073 [1980]); and other glycolytic enzymes (Hess et al., J. Adv. Enzyme Res. 7:149 [1968]; Holland et al., Biochemistry 17:4900 [1978]), e.g., enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In the constructions of suitable expression plasmids, the termination sequences associated with these genes are also ligated into the expression vector 3′ of the sequence desired to be expressed, to provide polyadenylation of the mRNA and termination. Other promoters that have the additional advantage of transcription controlled by growth conditions are the promoter regions for alcohol oxidase 1 (AOX1, particularly preferred for expression in Pichia), alcohol dehydrogenase 2, isocytochrome C, acid phosphatase, degradative enzymes associated with nitrogen metabolism, the aforementioned glyceraldehyde-3-phosphate dehydrogenase, and enzymes responsible for maltose and galactose utilization. Any plasmid vector containing yeast-compatible promoter and termination sequences, with or without an origin of replication, is suitable. Yeast expression systems are commercially available, for example, from Clontech Laboratories, Inc. (Palo Alto, Calif., e.g. pYEX 4T family of vectors for S. cerevisiae), Invitrogen (Carlsbad, Calif., e.g. pPICZ series Easy Select Pichia Expression Kit) and Stratagene (La Jolla, Calif. e.g. ESP™ Yeast Protein Expression and Purification System for S. pombe and pESC vectors for S. cerevisiae). The production of hVEGF₁₂₁ N75Q in P. pastoris is described in detail in the Examples below. Wild-type hVEGF₁₂₁ and other variants can be expressed in an analogous fashion.

Cell cultures derived from multicellular organisms may also be used as hosts to practice the present invention. While both invertebrate and vertebrate cell cultures are acceptable, vertebrate cell cultures, particularly mammalian cells, are preferable. Examples of suitable cell lines include monkey kidney cell line CV1 transformed by SV40 (COS-7, ATCC CRL 1651); human embryonic kidney cell line 293S (Graham et al, J. Gen. Virol. 36:59 [1977]); baby hamster kidney cells (BHK, ATCC CCL 10); Chinese hamster ovary (CHO) cells (Urlaub and Chasin, Proc. Natl. Acad. Sci. USA 77:4216 [1980]; monkey kidney cells (CV1-76, ATCC CCL 70); African green monkey cells (VERO-76, ATCC CRL-1587); human cervical carcinoma cells (HELA, ATCC CCL 2); canine kidney cells (MDCK, ATCC CCL 34); human lung cells (W138, ATCC CCL 75); and human liver cells (Hep G2, HB 8065). Expression of the VEGF₁₂₁ dimers herein in CHO cells is specifically illustrated in the examples.

Suitable promoters used in mammalian expression vectors are often of viral origin. These viral promoters are commonly derived from cytomegalovirus (CMV), polyoma virus, Adenovirus2, and Simian Virus 40 (SV40). The SV40 virus contains two promoters that are termed the early and late promoters. They are both easily obtained from the virus as one DNA fragment that also contains the viral origin of replication (Fiers et al., Nature 273:113 [1978]). Smaller or larger SV40 DNA fragments may also be used, provided they contain the approximately 250-bp sequence extending from the HindIII site toward the BglI site located in the viral origin of replication. An origin of replication may be obtained from an exogenous source, such as SV40 or other virus, and inserted into the cloning vector. Alternatively, the host cell chromosomal mechanism may provide the origin of replication. If the vector containing the foreign gene is integrated into the host cell chromosome, the latter is often sufficient.

Prokaryotes can also be used as host cells in producing the VEGF₁₂₁ dimers of the present invention. Suitable E. coli host strains include BL21; AD494 (DE3); EB105; and CB (E. coli B, ATCC 23848) and their derivatives; K12 strain 214 (ATCC 31,446); W3110 (ATCC 27,325); X1776 (ATCC 31,537); HB101 (ATCC 33,694); JM101 (ATCC 33,876); NM522 (ATCC 47,000); NM538 (ATCC 35,638); NM539 (ATCC 35,639), etc. Many other species and genera of prokaryotes may be used as well. Prokaryotes, e.g. E. coli, produce VEGF in an unglycosylated form.

Vectors used for transformation of prokaryotic host cells usually have a replication site, a marker gene providing for phenotypic selection in transformed cells, one or more promoters compatible with the host cells, and a polylinker region containing several restriction sites for insertion of foreign DNA. Plasmids typically used for transformation of E. coli include pBR322, pUC18, pUC19, pUC118, pUC 119, and Bluescript M13, all of which are commercially available and described in Sections 1.12-1.20 of Sambrook et al., supra. The promoters commonly used in vectors for the transformation of prokaryotes are the T7 promoter (see, e.g. U.S. Pat. Nos. 4,952,496 and 5,693,489 (Studier et al.)); the tryptophan (trp) promoter (Goeddel et al., Nature 281:544 [1979]); the alkaline phosphatase promoter (phoA); the β-lactamase and lactose (lac) promoters; and the bacteriophage λ p_{L} promoter systems.

In E. coli, the VEGF₁₂₁ monomers typically accumulate in the form of inclusion bodies, and need to be solubilized, refolded, dimerized and purified. Methods for the recovery and refolding of VEGF isoforms from E. coli are known in the art. For example, refolding of certain VEGF isoforms following recombinant expression in E. coli is described in Christinger et al., Prot. Struc. Func. Genet. supra (1996); Keyt et al., J. Biol. Chem. 271:7788-7795 (1996); Cao et al., J. Biol. Chem. 271:3154-3162 (1996); Siemeister et al., Biochem. Biophys. Res. Commun. 222:249-255 (1996); and PCT Publication WO 96/06641. In a particularly preferred embodiment of the present invention refolding is performed in the simultaneous presence of cysteine and cystine in the refolding buffer. By adjusting the amounts and mutual ratio of cysteine and cystine, one can produce the desired mix of VEGF dimers. The latter embodiment is specifically illustrated in the Examples below. In a preferred embodiment, free cysteine used in the refolding step is added in molar excess from about 4-fold to about 40-fold over the cysteines present in the VEGF polypeptide. More preferably, the free cysteine is used in from about 4-fold to about 20-fold, even more preferably from about 4-fold to about 10-fold, most preferably about 10-fold molar excess over the cysteines present in the VEGF polypeptide. Th cysteine to cystine molar ratio generally is between about 2:1 and 20:1, preferably between about 2:1 and 10:1, more preferably between about 2:1 and 5:1, most preferably about 4:1 and 5:1.

Prokaryotes, e.g. E.coli are known to remove the N-terminal (initiating) methionine (Met) from the primary translation product. As a result, protease(s) (aminopeptidases) present in the E.coli host cells may cleave residues from the N-terminus of the mature VEGF protein. To avoid this, in a preferred embodiment VEGF is expressed in E.coli with an N-terminal extension between the initiating Met and the mature N-terminus of the VEGF polypeptide. The extension usually comprises 1-7 identical or different amino acids, at least one of which is capable of retarding proteolytic degradation of the mature N-terminus. In a particularly preferred embodiment, the extension keeps the initiating Met intact during fermentation. In another embodiment Met and optionally part of the N-terminal extension are removed during the fermentation process, but at least a portion of the extension and, accordingly, the mature N-terminus remain intact. After recovering VEGF from the E. coli host cell, the extension can be removed, for example, by treatment with an aminopeptidases which has specificity that prevents its cleavage of the N-terminus of the VEGF molecule. Essentially the same approach can be adapted to situations when preservation of the mature N-terminus of other proteins is a problem during expression in E. coli.

Many eukaryotic proteins, including VEGF, contain an endogenous signal sequence as part of the primary translation product. This sequence targets the protein for export from the cell via the endoplasmic reticulum and Golgi apparatus. The signal sequence is typically located at the amino terminus of the protein, and ranges in length from about 13 to about 36 amino acids. Although the actual sequence varies among proteins, all known eukaryotic signal sequences contain at least one positively charged residue and a highly hydrophobic stretch of 10-15 amino acids (usually rich in the amino acids leucine, isoleucine, valine and phenylalanine) near the center of the signal sequence. The signal sequence is normally absent from the secreted form of the protein, as it is cleaved by a signal peptidase located on the endoplasmic reticulum during translocation of the protein into the endoplasmic reticulum. The protein with its signal sequence still attached is often referred to as the pre-protein, or the immature form of the protein, in contrast to the protein from which the signal sequence has been cleaved off, which is usually one of the steps necessary to create the mature protein. Proteins may also be targeted for secretion by linking a heterologous signal sequence to the protein. This is readily accomplished by ligating DNA encoding a signal sequence to the 5′ end of the DNA encoding the protein, and expressing the fusion protein in an appropriate host cell. Prokaryotic and eukaryotic (yeast and mammalian) signal sequences may be used, depending on the type of the host cell. The DNA encoding the signal sequence is usually excised from a gene encoding a protein with a signal sequence, and then ligated to the DNA encoding the protein to be secreted, e.g. VEGF. Alternatively, the DNA encoding the signal sequence can be chemically synthesized. The signal must be functional, i.e. recognized by the host cell signal peptidase and secretion pathway such that the signal sequence is cleaved and the protein is secreted. A large variety of eukaryotic and prokaryotic signal sequences is known in the art, and can be used in performing the process of the present invention. Yeast signal sequences include, for example, acid phosphatase, alpha factor, alkaline phosphatase, exo-1,3,-β-glucanase and invertase signal sequences. Prokaryotic signal sequences include, for example LamB, OmpA, OmpB and OmpF, MalE, PhoA, and β lactamase.

Mammalian cells are usually transformed with the appropriate expression vector using a version of the calcium phosphate method (Graham et al., Virology 52:546 [1978]; Sambrook et al., supra, sections 16.32-16.37), or, more recently, lipofection. However, other methods, e.g. protoplast fusion, electroporation, direct microinjection, etc. are also suitable.

Yeast hosts are generally transformed by the polyethylene glycol method (Hinnen, et al., Proc. Natl. Acad. Sci. USA 75:1929-1933 [1978]). Yeast, e.g. Pichia pastoris, can also be transformed by other methodologies, e.g. electroporation, as described in the Examples.

Prokaryotic host cells can, for example, be transformed using the calcium chloride method (Sambrook et al., supra, section 1.82), or electroporation.

If the host is Pichia pastoris, transformed cells can be selected for by using appropriate techniques including, but not limited to, culturing previously auxotrophic cells after transformation in the absence of the biochemical product required (due to the cell's auxotrophy), selection for and detection of a new phenotype, or culturing in the presence of an antibiotic which is toxic to the yeast in the absence of a resistance gene contained in the transformant. Isolated transformed Pichia pastoris cells are cultured by appropriate fermentation techniques such as shake flask fermentation, high density fermentation or the technique disclosed by Cregg et al. in, High-Level Expression and Efficient Assembly of Hepatitis B Surface Antigen in: The Methylotrophic Yeast, Pichia Pastoris, Bio/Technology 5:479-485 (1987). Isolates may be screened by assaying for VEGF₁₂₁ production to identify those isolates with the highest production level.

Transformed strains, that are of the desired phenotype and genotype, are grown in fermentors. For the large-scale production of recombinant DNA-based products in methylotrophic yeast, a three stage, high cell-density fed-batch fermentation system is normally the preferred fermentation protocol employed. In the first, or growth stage, expression hosts are cultured in defined minimal medium with an excess of a non-inducing carbon source (e.g. glycerol). If the expression vector is constructed such that expression of the desired product is driven by a promoter that is controlled by appropriate carbon source conditions, then heterologous gene expression can be completely repressed when the host is grown on the appropriate repressing carbon sources, which allows the generation of cell mass in the absence of heterologous protein expression. It is presently preferred, during this growth stage, that the pH of the medium be maintained at about 4.5-5. Next, a short period of non-inducing carbon source limitation growth is allowed to further increase cell mass and derepress the carbon source-responsive promoter. Subsequent to the period of growth under limiting conditions, the inducing carbon source, e.g., methanol, alone (e.g., “limited methanol fed-batch mode”) or a limiting amount of non-inducing carbon source plus inducing carbon source (referred to herein as “mixed-feed fed-batch mode”) is added in the fermentor, inducing the expression of the heterologous gene driven by the carbon source-responsive, e.g., methanol-responsive, promoter. This third stage is the so-called production stage. The pH of the medium during this production period is adjusted to between about pH 5 and about pH 6, preferably either about pH 5.0 or about pH 6.0. Expression of VEGF can also be conducted in shake flasks. By modifying the conditions during the production stage, e.g. by including cysteine, cystine and/or glutathione in the medium, the form of VEGF₁₂₁ dimer produced can be modulated such that the majority of the product is in a form containing a mixed disulfide at the Cys-116 position of each monomer subunit.

As we have found that the VEGF₁₂₁ dimers of the present invention are fully active, pharmaceutical compositions containing the dimers or product mixtures herein are within the scope of the present invention. Suitable forms, in part, depend upon the use or the route of entry, for example oral, transdermal, inhalation, implantation, or by infusion or injection. Such forms should allow the agent or composition to reach a target cell whether the target cell is present in a multicellular host or in culture. For example, pharmacological agents or compositions injected into the blood stream should be soluble. Other factors are known in the art, and include considerations such as toxicity and forms that prevent the agent or composition from exerting its effect under certain conditions.

Compositions comprising a VEGF₁₂₁ dimer or product mixture of the present invention can also be formulated as pharmaceutically acceptable salts (e.g., acid addition salts) and/or complexes thereof. Pharmaceutically acceptable salts are non-toxic at the concentration at which they are administered. Pharmaceutically acceptable salts include acid addition salts such as those containing sulfate, hydrochloride, phosphate, sulfonate, sulfamate, acetate, citrate, lactate, tartrate, methanesulfonate, ethanesulfonate, benzenesulfonate, p-toluenesulfonate, cyclohexylsulfonate, cyclohexylsulfamate and quinate. Pharmaceutically acceptable salts can be obtained from acids such as hydrochloric acid, sulfuric acid, phosphoric acid, sulfonic acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfonic acid, cyclohexylsulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying, or by exchanging the ions of an existing salt for another ion on a suitable ion exchange resin.

Carriers or excipients can also be used to facilitate administration of the dimers or product mixtures. Examples of carriers and excipients include calcium carbonate, calcium phosphate, various sugars such as lactose, glucose, sucrose or trehalose, or types of starch, cellulose derivatives, gelatin, vegetable oils, polyethylene glycols and physiologically compatible solvents. The compositions can be administered by different routes including, but not limited to, intravenous, intra-arterial, intraperitoneal, intrapericardial, intracoronary, subcutaneous, intramuscular, oral, topical, or transmucosal.

The desired isotonicity of the compositions can be accomplished using sodium chloride or other pharmaceutically acceptable agents such as dextrose, boric acid, sodium tartrate, propylene glycol, polyols (such as mannitol and sorbitol), or other inorganic or organic solutes.

Pharmaceutical compositions comprising a VEGF₁₂₁ dimer or a product mixture of the present invention can be formulated for a variety of modes of administration, including systemic and topical or localized administration. Techniques and formulations generally may be found in Remington's Pharmaceutical Sciences, 18th Edition, Mack Publishing Co., Easton, Pa. 1990. See, also, Wang and Hanson “Parenteral Formulations of Proteins and Peptides: Stability and Stabilizers”, Journal of Parenteral Science and Technology, Technical Report No. 10, Supp. 42-2S (1988). A suitable administration format can best be determined by a medical practitioner for each patient individually.

For systemic administration of a protein, injection is most commonly employed, e.g., intramuscular, intravenous, intra-arterial, intracoronary, intrapericardial, intraperitoneal, subcutaneous, intrathecal, or intracerebrovascular. For injection, the compounds of the invention are formulated in liquid solutions, preferably in physiologically compatible buffers such as Hank's solution or Ringer's solution. Alternatively, the compounds of the invention are formulated in one or more excipients (e.g., propylene glycol) that are generally accepted as safe as defined by USP standards. They can, for example, be suspended in an inert oil, suitably a vegetable oil such as sesame, peanut, olive oil, or other acceptable carrier. Preferably, they are suspended in an aqueous carrier, for example, in an isotonic buffer solution at pH of about 5.0 to 7.4. These compositions can be sterilized by conventional sterilization techniques, or can be sterile filtered. The compositions can contain pharmaceutically acceptable auxiliary substances as required to approximate physiological conditions, such as pH buffering agents. Useful buffers include for example, sodium acetate/acetic acid buffers and sodium citrate/citric acid buffers. A form of repository or “depot” slow release preparation can alternatively be used so that therapeutically effective amounts of the preparation are delivered into the bloodstream over many hours or days following implantation, injection or transdermal delivery. In addition, the compounds can be formulated in solid form and redissolved or suspended immediately prior to use. Lyophilized forms are also included.

The VEGF₁₂₁ dimers or product mixtures of the present invention can also be introduced directly into the heart, by using a catheter inserted directly into a coronary artery, as described, for example, in U.S. Pat. No. 5,244,460, or by using a catheter inserted into the ventricle of the heart to allow injection of the VEGF₁₂₁ dimers or product mixtures directly into the wall of the heart

Under certain circumstances, the dimers and product mixtures of the present invention may also be made available for oral administration. For oral administration, the dimers or product mixtures are formulated into conventional oral dosage forms such as capsules, tablets and tonics.

Systemic administration can also be by transmucosal or transdermal delivery. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents can be used to facilitate permeation. Transmucosal administration can be, for example, through nasal sprays or using suppositories.

For administration by inhalation, usually inhalable dry power compositions or aerosol compositions are used, where the size of the particles or droplets is selected to ensure deposition of the active ingredient in the desired part of the respiratory tract, e.g. throat, upper respiratory tract or lungs. Inhalable compositions and devices for their administration are well known in the art. For example, devices for the delivery of aerosol medications for inspiration are known. One such device is a metered dose inhaler that delivers the same dosage of medication to the patient upon each actuation of the device. Metered dose inhalers typically include a canister containing a reservoir of medication and propellant under pressure and a fixed volume metered dose chamber. The canister is inserted into a receptacle in a body or base having a mouthpiece or nosepiece for delivering medication to the patient. The patient uses the device by manually pressing the canister into the receptacle body to close a filling valve and capture a metered dose of medication inside the chamber and to open a release valve which releases the captured, fixed volume of medication in the dose chamber to the atmosphere as an aerosol mist. Simultaneously, the patient inhales through the mouthpiece to entrain the mist into the airway. The patient then releases the canister so that the release valve closes and the filling valve opens to refill the dose chamber for the next administration of medication. See, for example, U.S. Pat. No. 4,896,832 and a product available from 3M Healthcare known as Aerosol Sheathed Actuator and Cap.

Another device is the breath actuated metered dose inhaler that operates to provide automatically a metered dose in response to the patient's inspiratory effort. One style of breath actuated device releases a dose when the inspiratory effort moves a mechanical lever to trigger the release valve. Another style releases the dose when the detected flow rises above a preset threshold, as detected by a hot wire anemometer. See, for example, U.S. Pat. Nos. 3,187,748; 3,565,070; 3,814,297; 3,826,413; 4,592,348; 4,648,393; 4,803,978.

Devices also exist to deliver dry powdered drugs to the patient's airways (see, e.g. U.S. Pat. No. 4,527,769) and to deliver an aerosol by heating a solid aerosol precursor material (see, e.g. U.S. Pat. No. 4,922,901). These devices typically operate to deliver the drug during the early stages of the patient's inspiration by relying on the patient's inspiratory flow to draw the drug out of the reservoir into the airway or to actuate a heating element to vaporize the solid aerosol precursor.

Devices for controlling particle size of an aerosol are also known, see, for example, U.S. Pat. Nos. 4,790,305; 4,926,852; 4,677,975; and 3,658,059.

For topical administration, the compounds of the invention are formulated into ointments, salves, gels, or creams, as is generally known in the art.

If desired, solutions of the above compositions can be thickened with a thickening agent such as methyl cellulose. They can be prepared in emulsified form, either water in oil or oil in water. Any of a wide variety of pharmaceutically acceptable emulsifying agents can be employed including, for example, acacia powder, a non-ionic surfactant (such as a Tween), or an ionic surfactant (such as alkali polyether alcohol sulfates or sulfonates, e.g., a Triton).

Compositions useful in the invention are prepared by mixing the ingredients following generally accepted procedures. For example, the selected components can be mixed simply in a blender or other standard device to produce a concentrated mixture which can then be adjusted to the final concentration and viscosity by the addition of water or thickening agent and possibly a buffer to control pH or an additional solute to control tonicity.

The amounts of various dimers or product mixtures for use in accordance with the present invention can be determined by standard procedures. Generally, a therapeutically effective amount is between about 100 mg/kg and 10⁻¹² mg/kg depending on the age and size of the patient, and the disease or disorder associated with the patient. Generally, it is an amount between about 0.01 and 50 mg/kg, preferably 0.05 and 20 mg/kg, most preferably 0.05 and 2 mg/kg of the individual to be treated.

For use by the physician, the compositions are provided in dosage unit form containing an amount of a VEGF₁₂₁ dimer or mixture herein.

The VEGF₁₂₁ dimers and mixtures of the present invention are promising candidates for the same indications as other forms of VEGF. Accordingly, the VEGF₁₂₁ dimers and product mixtures herein can be used to induce angiogenesis and/or vascular remodeling, and therefore may find utility in the treatment of coronary artery disease and/or peripheral arterial disease. The VEGF₁₂₁ dimers and product mixtures of the present invention can be used, for example, to foster myocardial blood vessel growth and to improve blood flow to the heart (see, e.g. U.S. Pat. No. 5,244,460). Both peripheral arterial disease and coronary artery disease can often be treated successfully with either angioplasty/endarterectomy approaches (to open up the blockage caused by atherosclerotic plaque growth) or surgical bypass (to create a conduit around the blockage). In a significant number of cases, however, patients are deemed to be poor risks to be helped by either of these types of approaches (see, for example, Mukherjee et al., Am. J. Cardiol. 84:598-600 [1999]). It is this group of so-called “no option” patients that are expected to be the initial primary beneficiaries of the treatments provided by the present invention. It is foreseen that the new blood vessels, or newly-enlarged vessels, created in response to the treatment by the VEGF₁₂₁ dimers or product mixtures of the present invention, will create a natural bypass around the blocked vessels, without significant side-effects. As a result, the long-term hope is that this therapy will be used to replace angioplasty/endarterectomy/surgical bypass in the coronary artery disease and peripheral arterial disease patient populations in general, or at least in some cases.

The present invention is further directed to the treatment (including prevention) of injury to blood vessels and to the treatment (including prevention) of injury to tissues containing such blood vessels, in conditions where endothelial cell injury is mediated by known or unknown toxins, such as occurs in hemolytic uremic syndrome (HUS), toxic shock syndrome, exposure to venoms, or exposure to chemical or medicinal toxins, and in conditions where endothelial cell injury is mediated by hypertension.

The invention further concerns the treatment (including prevention) of kidney diseases associated with injury to, or atrophy of, the vasculature of the glomerulus and interstitium.

The invention also concerns the treatment (including prevention) of injury to the endothelium of blood vessels, and for the treatment (including prevention) of injury to tissues containing such injured blood vessels in diseases associated with hypercoagulable states, platelet activation or aggregation, thrombosis, or activation of proteins of the clotting cascade, preeclampsia, thrombotic thombocytopenic purpura (TTP), disseminated intravascular coagulation, sepsis, and pancreatis.

The invention also provides methods for the treatment (including prevention) of injury to blood vessels or injury to the surrounding tissue adjacent to injured blood vessels arising as a result of diminished blood flow due to decreased blood pressure, or full or partial occlusion of the blood vessel, due to atherosclerosis, thrombosis, mechanical trauma, vascular wall dissection, surgical dissection, or any other impediment to normal blood flow or pressure. Specifically, the invention provides methods for the treatment (including prevention) of acute renal failure, myocardial infarction with or without accompanying thrombolytic therapy, ischemic bowel disease, transient ischemic attacks, and stroke.

The invention also provides methods for the treatment (including prevention) of hypoxia or hypercapnia or fibrosis arising from injury to the endothelium of the lungs occasioned by injurious immune stimuli, toxin exposure, infection, or ischemia, including but not limited to acute respiratory distress syndrome, toxic alveolar injury, as occurs in smoke inhalation, pneumonia, including viral and bacterial infections, and pulmonary emboli.

The invention further provides methods and means for the treatment (including prevention) of pulmonary dysfunction arising from injury to the pulmonary endothelium, including disorders arising from birth prematurity, and primary and secondary causes of pulmonary hypertension.

The methods disclosed herein can also be used for the treatment of wounds arising from any injurious breach of the dermis with associated vascular injury.

The invention also provides methods for the treatment (including prevention) of injury to the endothelium and blood vessels, and for the treatment (including prevention) of injury to tissues containing injured blood vessels, due to injurious immune stimuli, such as immune cytokines, immune complexes, and proteins of the complement cascade, including but not restricted to diseases such as vasculitis of all types, allergic reactions, diseases of immediate and delayed hypersensitivity, and autoimmune diseases.

Specific kidney diseases that may be treatable by using the methods of the present invention include HUS, focal glomerulosclerosis, amyloidosis, glomerulonephritis, diabetes, SLE, and chronic hypoxia/atrophy.

The VEGF₁₂₁ dimers and product mixtures of the present invention can also be used for treating or preventing hypertension. Effectiveness of the treatment is determined by decreased blood pressure particularly in response to salt loading.

The VEGF₁₂₁ dimers and product mixtures of the present invention can also be useful in treating disorders relating to abnormal transport of solutes across endothelial cells. Such disorders include (1) kidney disease associated with impaired filtration or excretion of solutes; (2) diseases of the central nervous system associated with alterations in cerebrospinal fluid synthesis, composition, or circulation, including stroke, meningitis, tumor, infections, and disorders of spinal bone growth; (3) hypoxia or hypercapnia or fibrosis arising from accumulation of fluid secretions in the lungs or impediments to their removal, including but not restricted to acute respiratory distress syndrome, toxic alveolar injury, as occurs in smoke inhalation, pneumonia, including viral and bacterial infections, surgical intervention, cystic fibrosis, and other inherited or acquired disease of the lung associated with-fluid accumulation in the pulmonary air space; (4) pulmonary dysfunction arising from injury to the pulmonary endothelium, including disorders arising from birth prematurity, and primary and secondary causes of pulmonary hypertension; (5) diseases arising from disordered transport of fluid and solutes across the intestinal epithelium, including but not restricted to inflammatory bowel disease, infectious diarrhea, and surgical intervention; and (6) ascites accumulation in the peritoneum as occurs in failure of the heart, liver, or kidney, or in infectious or tumor states. Additional uses include: (1) the enhancement of efficacy of solute flux as it can be needed for peritoneal dialysis in the treatment of kidney failure or installation of therapeutics or nutrition into the peritoneum; (2) the preservation or enhancement of function of organ allografts, including but not restricted to transplants of kidney, heart, liver, lung, pancreas, skin, bone, intestine, and xenografts; and (3) the treatment of cardiac valve disease.

Further details of the present invention will be apparent from the following non-limiting Examples. All references cited throughout the specification, including the Examples, are hereby expressly incorporated by reference.

### EXAMPLES

### Example 1

### Production of hVEGF₁₂₁ in Mammalian Host Cells

A. Generation of Cell Lines Producing hVEGF₁₂₁

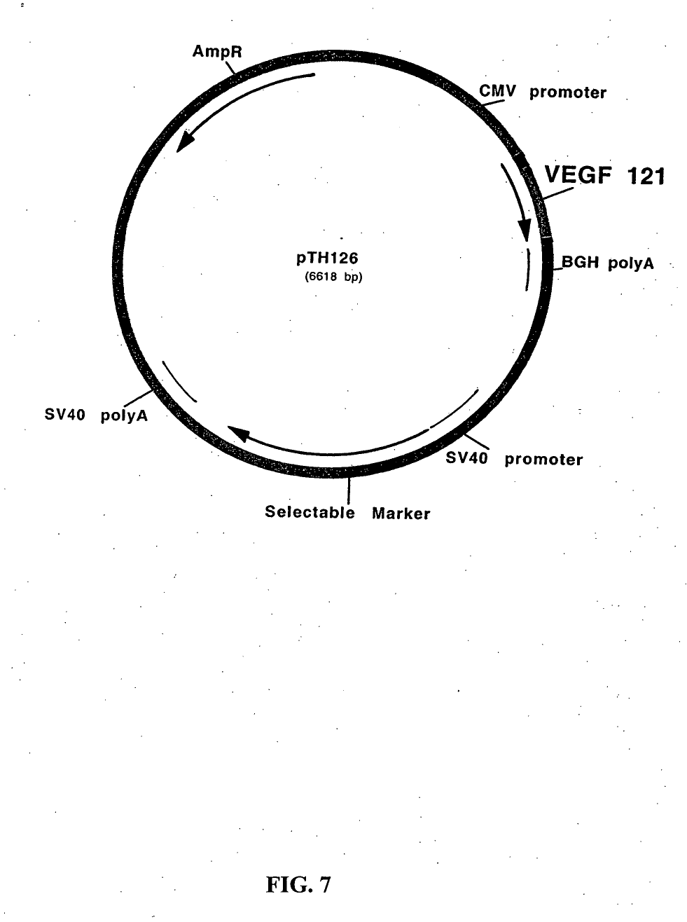

Vector: A plasmid expression vector (FIG. 7) was created in which the cDNA encoding hVEGF₁₂₁ precursor (secretion signal+mature 121-residue monomer chain) was operably linked to a highly active promoter, derived from the cytomegalovirus (CMV) middle later promoter. The transcription termination/polyadenylation region from the bovine growth hormone gene was placed downstream of the VEGF cDNA. The expression plasmid also encodes a protein that can be used for selection and amplification of the plasmid once it has been introduced into mammalian cells. Suitable selectable markers include dihydrofolate reductase (DHFR) and glutamine synthetase, but other common selectable markers are just as suitable. Expression of the selectable marker is driven by the SV40 early promoter, and an SV40 transcription termination/polyadenylation signal is located downstream of the marker. To allow propagation in bacterial cells, the vector also contains a bacterial (ColEI) origin of replication and encodes β-lactamase, which imparts ampicillin resistance.

Selection of CHO Cell Lines Expressing VEGF₁₂₁: LipofectAMINE (GIBCO-BRL) was used to introduce the VEGF expression vector into 70% confluent Chinese Hamster Ovary (CHO) cells (CHO-K1, obtained from ATCC; or, if DHFR is the selectable marker, CHO DG44 (dhfr⁻) cells, obtained from Laurence Chasin, Columbia University, New York, N.Y.). After 24 hours of recovery in a 50:50 (v/v) mix of DMEM (high glucose) and Coon's F12 medium, the cells were trypsinized, centrifuged, and then resuspended and plated in a selective medium. In the case of DHFR selection, the selective medium was IMDM supplemented with 2% dialyzed fetal bovine serum (JRH Biosciences) and 1 ×SITE (selenite, insulin, transferrin, and ethanolamine; Sigma). With glutamine synthetase as the selectable marker, the selective medium was glutamine-free DMEM (high glucose) containing 1×GS supplement (JRH Biosciences, Lenex, Kans.), 10% dialyzed fetal bovine serum, and 25 μM methionine sulfoximine. The population of cells that survived in the selective medium was collected by trypsinization and replated into multiple 96-well plates. Individual plates of the cells were then treated with selective medium containing either increasing concentrations (over time) of methotrexate (if DHFR was the selection marker), or various concentrations of the methionine sulfoximine selective agent (200 μM, 400 μM, or 600 μM), if glutamine synthetase was the marker. After 11 days of selection/amplification, samples of conditioned media from the wells were collected and tested for level of VEGF expression by Western dot-blotting, using a rabbit polyclonal antibody raised against a VEGF peptide, or using a sandwich ELISA kit (R&D Systems, Minneapolis, Minn.). One clone showing the highest level of expression for a given selectable marker was chosen for use in producing recombinant hVEGF₁₂₁.

B. Production of Recombinant hVEGF₁₂₁

Production of Conditioned Medium from CHO Cell Line Expressing VEGF₁₂₁: The CHO cell clone was propagated in one of two different media. For cells in monolayer culture, a 50:50 mix of DMEM-21 and Coon's F12 (both glutamine-free) was used that was supplemented with 10% dialyzed fetal bovine serum and either 80 nM methotrexate and 4 mM glutamine (for a clone containing a DHFR selectable marker) or 100 μM methionine sulfoximine (if glutamine synthetase was the marker). Alternatively, if the cells were in suspension culture, the medium was ProCHO4 CD4 from Biowhitikar (Walkersville, Md.), supplemented with 4 mM glutamine and 80 nM methotrexate (for a DHFR system clone) or 100 μM hypoxanthine, 16 μM thymidine, and 100 μM methionine sulfoximine (for a glutamine synthetase system clone). For monolayer culture, confluent T225 flask cultures were trypsinized, collected by centrifugation, and plated into 1700 cm² roller bottles. Each roller bottle received the equivalent of one or two T225 flasks' worth of cells. The cells in the roller bottles were allowed to grow to confluence. The growth medium at this stage was supplemented with 15-20 mM HEPES (pH 7.2-7.5). When the cells reached confluence, the medium was removed, and the adherent cells were washed with phosphate-buffered saline. Serum-free medium (Ex-Cell PF-325 medium from JRH Biosciences, supplemented with 15-20 mM HEPES, pH 7.2-7.5) was then added to each roller bottle. The medium was collected from the roller bottles every 2-3 days, and replaced with fresh medium. The collected medium was filtered through a 0.22 μm filter, supplemented with 0.1 mM phenylmethylsulfonyl fluoride, and frozen.

C. Purification of hVEGF₁₂₁ from the Roller Bottle Conditioned Medium.

In some instances, the thawed conditioned medium was concentrated prior to fractionation; in other cases the thawed medium was used without concentration. In either case, the medium was applied to a DEAE Sepharose column that had been equilibrated in 10 mM Tris, pH 7.5. Bound protein was eluted with a gradient of NaCl (0 to 300 mM) in 10 mM Tris, pH 7.5. Fractions containing hVEGF₁₂₁ were pooled and applied to a Zn-Sepharose column that had been equilibrated with 10 mM Tris, pH 7.5, 0.5 M NaCl, 0.5 mM imidazole. The column was washed with equilibration buffer, or equilibration buffer supplemented to contain a total of 20 mM imidazole. Bound proteins were then eluted with a gradient of imidazole (either 0-60 mM, or 20-60 mM) in 10 mM Tris, pH 7.5, 0.5 M NaCl. Generally, two peaks of material containing VEGF were obtained. These peaks were each concentrated by ultrafiltration and fractionated further using a reversed-phase HPLC column (either C4 or C18) equilibrated in 25% acetonitrile, 0.1% trifluoroacetic acid. After each protein sample was loaded onto the column, the column was washed with equilibration buffer, and bound protein was eluted with a gradient of acetonitrile (25-45%) in 0.1% trifluoroacetic acid. Using the C4 column to purify hVEGF₁₂₁, one peak of VEGF was obtained from each Zn-Sepharose peak loaded on the column. When a C18 column was used, generally two VEGF peaks were obtained from each Zn-Sepharose sample.

D. Characterization of Recombinant hVEGF₁₂₁

Amino-terminal Sequencing Using the Applied Biosystems 494 Procise Protein Sequencer. N-terminal sequencing indicated that 90-95% of the VEGF₁₂₁ generated by the CHO cells begins with the correct sequence of native human VEGF₁₂₁ (Ala-Pro-Met-Ala-Glu . . . ). Molecules starting with residue 3 (Met), 4 (Ala) or 11 (His) have also been detected. In a representative case, the N-termini were about 90% residue 1, about 8% residue 4, and about 2% residue 11. In general, the product produced in CHO cells, is typically a mixture containing about 90-95% of a product starting with residue 1 (the correct N-terminus of the native molecule), about 3-10% of a product starting with residue 4, and about 0-2% of a product starting with residue 11 of the native molecule.

Mass Spectrometry Coupled with Liquid Chromatography (LC-MS) Using an LC2 Mass Spectrometer (Finnegan). LC-MS provides information on the masses of the molecules contained in the RP-HPLC fractions. From this information, one can deduce (1) whether the C-terminus of the molecule is intact, and (2) whether the VEGF molecule has been modified through covalent attachment—i.e., by glycosylation, or by disulfide bonding to other molecules (like cysteine). One also gets information on the structure of the glycosylation. According to LC-MS results, essentially all of the hVEGF₁₂₁ produced in CHO cells was found to end with residue 120, missing the final Arg residue in the native human sequence, although this loss varied somewhat with conditions. In certain preparations, up to about 65-70% of the hVEGF₁₂₁ molecules retained residue 121 of the native protein. The LC-MS data also showed that the VEGF monomers within the VEGF₁₂₁dimers were sometimes glycosylated and sometimes not. When the monomers were glycosylated, the N-linked sugar was found to have either one or two sialic acid moieties. Finally, the LC-MS data suggested that in some cases, two extra (extraneous) cysteine molecules had become bonded to the VEGF dimer (i.e., the molecular weight was increased by 240 atomic mass units [amu], consistent with the addition of two cysteines).

E. Confirmation of the C-terminus and the State of Cys-116 Using Glu-C Digestion.

Glu-C will cut proteins after glutamic acid (Glu) residues. In the case of hVEGF₁₂₁ dimers, since the middle of the molecule is tied up in a “cysteine knot” that makes it inaccessible to proteases, the only clips that Glu-C will make are after residue 5, residue 13, and residue 114. The cut at residue 114 of the CHO-derived hVEGF₁₂₁ liberates a C-terminal fragment representing residues 115-120 (or 115-121, if the molecule is full-length). This fragment can be completely sequenced by N-terminal sequencing, to determine whether essentially all of the molecules end at residue 120, or if any of the molecules contain residue 121. In addition, if the Cys at residue 116 is disulfide-bonded to another cysteine, the N-terminal sequencing will show a cystine (Cys-S-S-Cys) residue at cycle 2. LC-MS analysis of the Glu-C digest provides the mass of the C-terminal peptide. This mass can confirm loss of residue 121. In addition, this mass clearly distinguishes between a number of different states for Cys-116. If Cys-116 has become disulfide-bonded to an additional extraneous cysteine molecule, then the mass of the C-terminal Glu-C peptide will represent residues 115-120, plus 120 amu (for a total mass of 865 amu). If, on the other hand, Cys116 has become disulfide-bonded with the other Cys116 in the VEGF dimer molecule, then the C-terminal Glu-C fragment will contain residues 115-120 from both chains of the VEGF dimer, joined through the Cys116-Cys116 disulfide bond (for a total mass of 1490). If the arginine residue at position 121 has been retained, the masses of the possible C-terminal fragments will be 1021 and 1802, respectively.

For the proteolytic fragmentation, VEGF (0.2-1.5 mg/ml) in phosphate-buffered saline (adjusted to pH 5.5 with citric acid) was digested at 37° C. for 24 hours with Glu-C (Boehringer Mannheim) at an enzyme to substrate ratio of 1:25. Another aliquot of Glu-C at an enzyme to substrate ratio of 1:25 was then added, and the reaction was allowed to proceed at 37° C. for an additional 24 hours. The digestion products were then either applied to the protein sequencer or subjected to LC/MS. The results confirmed that in the hVEGF₁₂₁ dimers generated as described in Section B above, the Arg at position 121 was lost, and Cys-116 was sometimes disulfide bonded to an extraneous cysteine and sometimes bonded to the other Cys-116 in the dimer.

### Example 2

### Production of hVEGF₁₂₁ in E. coli Host Cells

A. E. coli Expression Plasmid

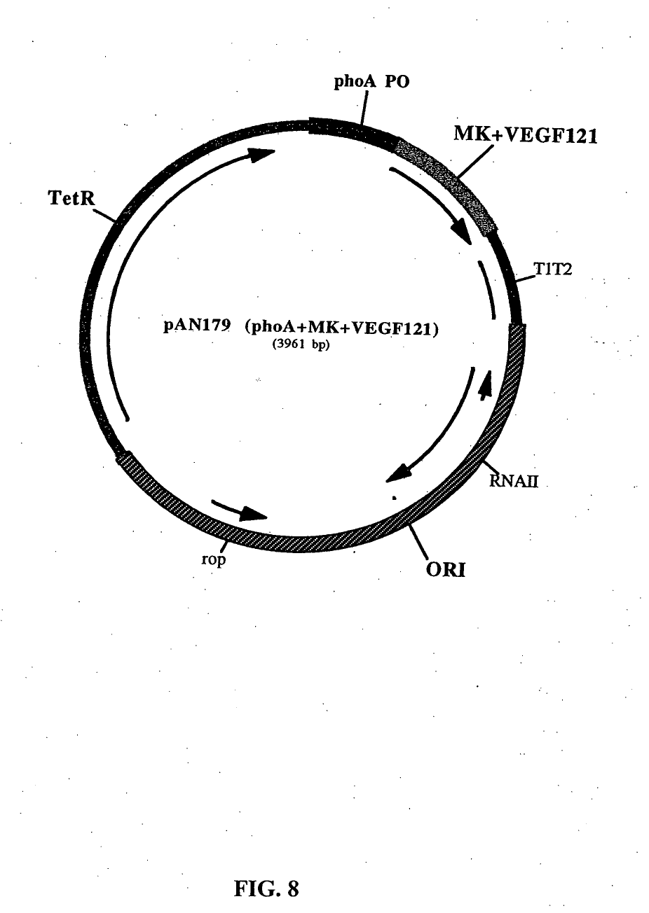

Expression of hVEGF₁₂₁ in E. coli host cells was accomplished using the expression vector pAN179 (FIG. 8). To create this plasmid, a synthetic coding sequence for hVEGF₁₂₁ was first created that reflected the codon biases seen in highly expressed E. coli genes. This coding sequence also incorporated two additional in-frame codons (a methionine codon and a lysine codon) at its 5′ end, so that the encoded product was 123 amino acids in length (“MK+VEGF₁₂₁”). The methionine codon was added to provide a translation initiation codon operative in E. coli. The lysine encoded by the second codon served to retard protease digestion of the hVEGF₁₂₁ product during synthesis in, and recovery from, the host cells. The coding sequence for MK+VEGF₁₂₁ was operably linked to a phoA promoter/operator (PO) region, so that transcription of the coding sequence could be initiated by depletion of phosphate in the growth medium. The T1T2 region of the E. coli rrnB locus was placed downstream of the coding sequence to provide transcription termination. The origin of replication (ORI) region for pAN179 was taken from pBR322, and retained the rop gene. A tetracycline resistance gene was also incorporated into the vector, to enable selection for plasmid presence and stability. The completed pAN179 plasmid was transformed into E. coli B cells (ATCC 23848), and a single-cell clone containing the plasmid was isolated by tetracyline selection on agar plates.

B. Production of Recombinant MK+VEGF₁₂₁ in E. coli by Fed-batch Fermentation

The E.coli B clone containing pAN179 was used to inoculate 25 mL of E. coli tank medium (Table 1) supplemented with 1% (w/v) glycerol and 1% (w/v) casamino acids. After incubation with shaking at 30° C. overnight, 5 mL of the resulting culture was used to inoculate 500 mL of the supplemented E. coli tank medium in a Fembach flask. The flask was incubated overnight with shaking at 30° C., and the entire culture was then added to a 10-L fermentor containing 8 L of E. coli tank medium (Table 1). The temperature of the fermentation was controlled at 30° C. The culture was agitated using an impeller rotation rate of 1000 rpm, and was aerated at 10.0 L/min. The pH of the culture was maintained at 6.7 with additions of 2 N hydrochloric acid and 14.8 M ammonium hydroxide. Antifoam was added as needed. After approximately 3.5-5.5 hours of batch growth, the glycerol in the medium had been exhausted as evidenced by a rapid rise in the dissolved oxygen (DO) level in the fermentation culture. The rise in dissolved oxygen level triggered the initiation of a glycerol feed, which was added at a controlled rate to maintain the DO level at 25% of saturation (with the limitation that the feed could not exceed 120 mL/hr). The glycerol feed consisted of 1021 g/L glycerol, 20 g/L magnesium sulfate heptahydrate, and 10 mL/L Korz Feed Trace Minerals (Korz et al, J. Bacteriol. 39:59-65, [1995]). After approximately 9-11 hours, potassium dihydrogen phosphate (32.5 g/L solution) was fed into the culture at a rate of approximately 6 g/hr to prevent the deleterious effects of phosphate starvation. This phosphate feed was continued until the end of the fermentation. After about 72 hours, the cells were harvested by centrifugation and frozen.

TABLE 1
E. coli Tank Medium

| Ingredient | Amount | |
|---|---|---|
| H₂O | 6.4 | L |
| (NH₄)₂SO₄ | 29.0 | g |
| (NH₄)₂HPO₄ | 5.9 | g |
| KH₂PO₄ | 20.0 | g |
| Citric Acid (anhydrous) | 13.6 | g |
| Casamino Acids | 80.0 | g |
| Glycerol | 40.0 | g |
| MgSO₄.7H₂O | 9.60 | g |
| Dissolve components completely, then add | | |
| Korz Tank Trace Elements | 80.0 | mL |
| (as in Korz et al., J. Bacteriol. 39: 59-65, | | |
| 1995, except no thiamine-HCl was added) | | |
| Adjust pH to 6.3 (with NaOH) | | |
| Sterilize in fermentor, cool to 30° C., | | |
| adjust volume to 8.0 L, then add | | |
| Tetracycline (10 mg/mL solution) | 8.0 | mL |

C. Purification of E. coli-derived hVEGF₁₂₁ Dimers

1. Isolation of the MK+VEGF₁₂₁ Monomer

During the fermentation, the MK+VEGF₁₂₁ product was deposited by the cells into insoluble inclusion bodies. To recover these inclusion bodies, the cell paste from the fermentation was first thawed and resuspended in deionized water. This suspension was centrifuged, the supernatant solution was discarded, and the pellet was suspended to a density of 15-20% (wet weight/volume) in lysis buffer (50 mM ethylenediamine, 150 mM NaCl, 5 mM EDTA, pH 6.5). The cells were then lysed by passage through an APV Gaulin 30CD high-pressure homogenizer set to 10,000 psi. Five continuous volumetric passes were performed to assure nearly complete lysis of the cells to release the inclusion bodies. The temperature of the lysate was maintained at <15° C. by flowing the lysate through a cooling coil and keeping the cell and lysate reservoir on ice. Inclusion bodies were separated from the cell debris and from soluble components by centrifugation (4000×g for 30 minutes). The pellet of inclusion bodies was washed by resuspension in lysis buffer followed by agitation for 16 hours at 2-8° C. The inclusion bodies were again collected by centrifugation, and were then resuspended in lysis buffer to 30% solids (wet weight/volume). The inclusion body suspension was stored frozen at −70° C. in aliquots.

For solubilization, the frozen inclusion bodies were first thawed, diluted 1:5 with lysis buffer, and then collected by centrifugation. The inclusion body pellet was dissolved in 7M urea, 20 mM Tris, 100 mM dithiotreitol (DTT), pH 7.8. The mixture was stirred under nitrogen at room (ambient) temperature (18-22° C.) for 3 hours. The solubilized material was then adjusted to 25 mM acetic acid (final concentration), and HCl was added until the pH of the solution was 4. The adjusted mixture was then filtered to 1.2 μm through a depth filter (Sartorius, Göttingen, Germany).

The filtered solution was diluted 1:5 with SP-1 equilibration buffer (6M urea, 25 mM sodium acetate, 5 mM DTT, pH 4), and then loaded onto a SP Sepharose Fast Flow (Amersham-Pharmacia Biotech, Uppsala, Sweden) chromatography column. The UV absorbance of the column eluate was monitored at 280 nm. The loaded column was washed with buffer containing 6M urea, 25 mM sodium acetate, 5 mM cysteine, 100 mM NaCl, pH 4. The reduced MK+VEGF₁₂₁ monomer was eluted from the column with the wash buffer supplemented to contain 550 mM NaCl. Fractions containing MK+VEGF₁₂₁ monomer were pooled.

2. Formation and Purification of hVEGF₁₂₁ Dimer

The pool of fractions from the SP Sepharose Fast Flow column (SP-1 pool) was diluted to 0.5 mg/mL reduced MK+VEGF₁₂₁ and adjusted to 2M urea, 25 mM diethanolamine, 400 mM NaCl, 2.5 mM cysteine, 0.55 mM cystine, pH 8.8. The resulting mixture was transferred to a stainless steel tank and stirred under ambient conditions for 41 hours to allow for oxidation of the cysteine residues in the protein by disulfide bond formation. Samples taken at various timepoints during the refolding reaction were subjected to reverse-phase HPLC fractionation followed by mass spectrometry. These analyses indicated that the course of MK+VEGF₁₂₁ refolding and dimerization followed a progression: at early timepoints, the molecular masses of the two predominant dimer forms were consistent with (1) a dimer in which a disulfide bond was present between the two Cys-116 residues in the dimer, and (2) a dimer with free sulfhydryl groups at the Cys-116 positions. At later times (e.g., at the end of the 41-hour stirring period), the primary dimer form had a molecular mass that was larger than the major early-timepoint dimers by approximately 240 amu, consistent with the presence of an additional cysteine moiety disulfide-bonded at each of the two Cys-116 positions. At intermediate times, substantial amounts of a form containing only one additional cysteine (i.e., mass increased by 120 amu) were detected. Hence, it was possible to manipulate the proportions of the dimer forms present in the refolding reaction by manipulating the time that the reaction was allowed to proceed. Pilot experiments indicated that the specific dimer form mix could also be manipulated by altering the ratio of reduced to oxidized cysteine present in the initial refolding mix.

After 41 hours of stirring in the steel tank, the refolding mixture was adjusted to 20 mM sodium phosphate and pH 7.7, and then filtered to 0.2 μm (Millex GP-50 filter, Millipore, Bedford, Mass.). The refolded MK+VEGF₁₂₁ dimers were captured on a zinc-loaded Chelating Sepharose Fast Flow (Amersham-Pharmacia) column. The UV absorbance of the eluate from this column was monitored at 280 nm. The loaded column was washed with 20 mM sodium phosphate, 200 mM NaCl, pH 7.7 buffer to remove unbound protein. Bound MK+VEGF₁₂₁ dimer was eluted from the column with 50 mM sodium acetate, 200 mM NaCl, pH 4. A single fraction containing MK+VEGF₁₂₁ dimer was collected. This fraction was adjusted to 1 mM EDTA and pH 5.0, and diaminopeptidase-1 (activated HT-DAP-1 enzyme, Unizyme, Denmark) was added at a weight ratio of 1:2000 (HT-DAP-1: total protein). The mixture was stirred under nitrogen at ambient temperature for 5 hours. The course of the conversion of MK+VEGF₁₂₁ dimer to hVEGF₁₂₁ dimer was followed by ion-exchange HPLC. The efficiency of the conversion and the N-terminal sequence were confirmed by automated Edman degradation peptide sequencing.

The reaction mixture resulting from the HT-DAP-1 cleavage reaction was diluted to 1 mg/mL protein and adjusted to 0.9 M ammonium sulfate, 25 mM sodium acetate, pH 4. After filtration to 0.2 μm (Millex GP-50 filter, Millipore), the mixture was applied to a column of Toyopearl Butyl-650M (TosoHaas, Montgomeryville, Pa.). Protein bound to the column was washed with 25 mM sodium acetate, 1.0 M ammonium sulfate, pH 4, and was then step-eluted with buffers of 25 mM sodium acetate, pH 4, containing 0.7 M, 0.3 M, and 0.15 M ammonium sulfate. The UV absorbance of the column eluate was monitored at 280 nm. Fractions were collected from each step and assayed by reverse-phase HPLC for the presence of the desired hVEGF₁₂₁ dimer form containing two additional cysteine moieties. Fractions containing a high proportion of this desired hVEGF₁₂₁ dimer were pooled. Ultrafiltration was performed using a Pellicon XL Biomax-5 membrane cassette (Millipore) to concentrate the pooled fractions. The resulting solution was diluted with sodium acetate buffer (50 mM, pH 4) to reduce the conductivity of the solution to a level compatible with hVEGF₁₂₁ dimer protein binding to the final column step of the purification (SP-5PW Ion Exchange Chromatography)

The diluted pool from the Toyopearl Butyl column chromatography was applied to a SP-5PW 30 μm resin (TosoHaas) column that had been equilibrated in 30 mM sodium acetate, 100 mM NaCl, pH 5.0. The UV absorbance of the column eluate was monitored at 280 nm. After loading, the column was washed with equilibration buffer, and bound protein was then eluted with a linear gradient of 100 to 300 mM NaCl in 50 mM sodium acetate, pH 5.0. Fractions were assayed for hVEGF₁₂₁ dimer content and purity by ion-exchange HPLC. Fractions containing hVEGF₁₂₁ dimer (form with two additional cysteines) at the desired purity were pooled, and the buffer was exchanged by ultrafiltration/diafiltration into 20 mM sodium citrate, 1 mM EDTA, 9% (w/v) sucrose, pH 5.0, using the Pellicon XL Biomax-5 ultrafiltration device and Labscale TFF system (Millipore). The solution was filtered to 0.2 μm (Sterivex-GP filter, Millipore), and then frozen at −70° C.

D. Analysis of E. coli-derived hVEGF₁₂₁ Dimer Product

The mass of the final product was determined by LC-MS analysis. This analysis in addition probed whether other forms of hVEGF₁₂₁ dimer were present in the final mix. The LC-MS data indicated that two forms of the molecule were present in the product: a major form with a mass of 28,365 amu (the predicted mass for the hVEGF₁₂₁ dimer containing amino acids 1-121, plus two additional cysteine moieties); and a minor form with a mass of 28,134 amu (consistent with the predicted mass for the hVEGF₁₂₁ dimer containing amino acids 1-121 and no additional cysteines). Reverse-phase HPLC analysis also showed the presence of these two forms in the product, and indicated that the forms were present in relative concentrations of about 93% higher mass form and 7% lower mass form. SDS-PAGE confirmed that the product was primarily in the form of a dimer. Amino-terminal amino acid sequencing demonstrated that 96-97% of the product initiated with the expected sequence (Ala-Pro- . . . ). The remainder of the product initiated at residue -2 (Met-Lys-Ala-Pro- . . . ; 0.8-1%), residue -1 (Lys-Ala-Pro- . . . ; 0.4-0.7%), or residue 5 (Glu-Gly-Gly-Gly . . . ; 1.6-1.7%). Thermolysin digestion followed by LC-MS confirmed the presence of additional cysteine moieties bonded to the cysteine residues at position 116 in the majority of the hVEGF₁₂₁ product.

### Example 3

### Production of hVEGF₁₂₁ in Pichia pastoris

A. Generation of P. pastoris Cell Line Producing hVEGF₁₂₁ N750

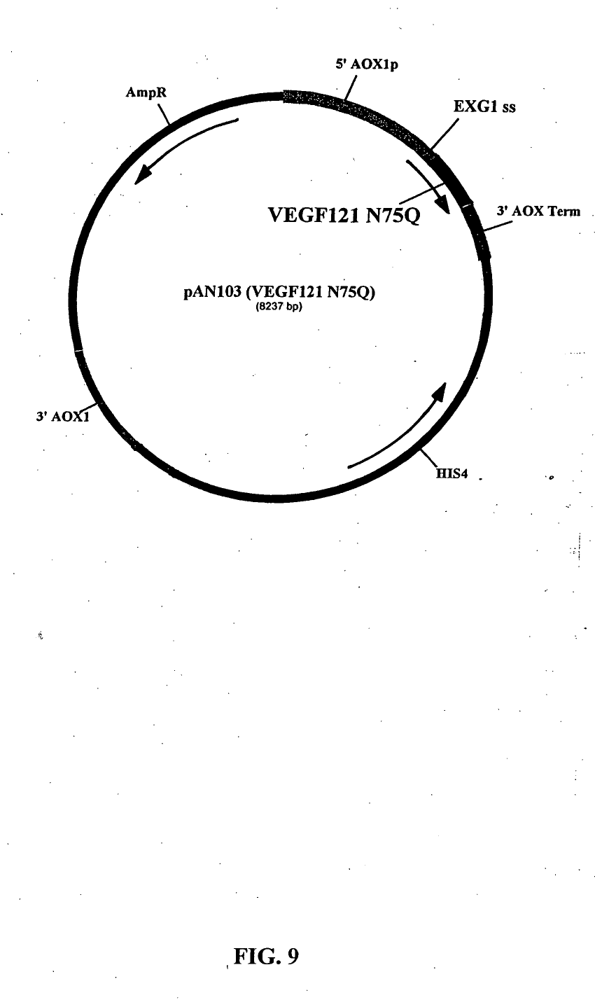

Vector: The plasmid expression vector (pAN103) created to direct expression of hVEGF₁₂₁ in P. pastoris is shown in FIG. 9. The cDNA encoding the 121 amino acids of the mature hVEGF₁₂₁ monomer primary structure was modified at codon 75 so that the amino acid encoded at this position was changed from asparagine to glutamine. The resulting cDNA thus encoded an N75Q variant form of VEGF₁₂₁. This change was made to eliminate the site of N-linked glycosylation found in the wild-type VEGF monomer sequence at residue 75. The altered cDNA sequence was then fused in-frame at its 5′ end to a DNA sequence (“EXG1 ss”) encoding the secretion signal sequence of the Saccharomyces cerevisiae exo-1,3-β-glucanase protein. In pilot experiments, this signal sequence was found to be more efficacious than the native human VEGF signal sequence at effecting secretion of the recombinant hVEGF₁₂₁ product from the P. pastoris host cells. The pilot experiments additionally indicated that the signal sequence encoded by the S. cerevisiae alpha factor gene could also be used to drive secretion of hVEGF₁₂₁ from P. pastoris. In pAN103, the hybrid cDNA (encoding the fusion protein joining the EXG1 signal sequence to the VEGF₁₂₁ monomer sequence) was operably linked to the promoter (“5′ AOX1p”) for the P. pastoris alcohol oxidase 1 (AOX1) gene. Transcription initiating from the AOX1 promoter is low to undetectable when P. pastoris is grown on glucose or glycerol, but is dramatically up-regulated when the cells are given methanol as the carbon source. The 3′ end of the AOX1 gene (“3′ AOX Term”) was placed downstream of the hybrid cDNA in order to provide transcription termination signals. The vector also carried the wild-type P. pastoris gene encoding histidinol dehydrogenase (HIS4), to allow selection for the plasmid in his4 host cells. In addition, the vector encoded ampicillin resistance and carried a ColE1 origin of replication to allow for manipulation in E. coli prior to introduction into P. pastoris host cells.

Selection of P. pastoris Cell Line Expressing hVEGF₁₂₁ N75Q: Plasmid pAN103 was digested with SalI, which cleaved the plasmid once within the HIS4 sequence. The resulting linear DNA was transformed by electroporation into P. pastoris mut+ (methanol utilization proficient) strain GS115. Cells were selected for acquisition of histidine prototrophy by plating on solid agar medium lacking histidine (RDB plates [18.6% (w/v) sorbitol, 2% (w/v) glucose, 1.34% (w/v) yeast nitrogen base, 0.4 μg/ml biotin, 2% (w/v) agar]) and incubating at 30° C. To assure that the genomic copy of AOX1 had not been disrupted, the colonies were also checked for the ability to grow on minimal methanol plates at 30° C. To check for expression of secreted hVEGF₁₂₁, single colonies obtained from the RDB plates were first inoculated into 2 ml buffered minimal glycerol YE/Peptone (BMGY) medium and grown with shaking at 30° C. overnight. Cells in each of the cultures were collected by centrifugation and resuspended in buffered minimal methanol YE/Peptone (BMMY) medium, and were then incubated in a 30° C. shaker for 48 hours to allow for induction of hVEGF₁₂₁ expression. To measure the level of hVEGF₁₂₁ produced, aliquots of the cell culture supernatants were analyzed by dot-blot, enzyme-linked immunosorbant assay (ELISA), and/or sodium dodecyl sulfate polyacrylamide gel electrophoresis (SDS-PAGE) followed by protein staining or Western blotting. Anti-human VEGF antibody (R&D Systems, Minneapolis, Minn.) was used as per the manufacturer's specifications to detect the product in the dot-blot and Western analyses. The ELISA kit used was also obtained from R&D Systems. Based on these analyses, one clone (ABL189) was chosen for use in larger-scale production of hVEGF₁₂₁.

B. Production of Recombinant hVEGF₁₂₁ N75Q by Fed-batch Fermentation Process

The process of producing a fermentation batch of hVEGF₁₂₁ N75Q was initiated by inoculating a 25-50 mL culture of YYG phosphate medium either with a single colony from a streak plate of P. pastoris strain ABL189, or with 25 μL from a thawed storage vial of ABL189 cells. The YYG phosphate medium consisted of 1% (w/v) yeast extract, 1.34% (w/v) yeast nitrogen base, 0.4 μg/mL biotin, 2% (v/v) glycerol, and 0.125 M phosphate buffer, pH 6.0. The culture was incubated in a baffled, 250- or 500-mL shake flask overnight at 30° C. with shaking. An aliquot of the culture was then used to inoculate 250 mL of YYG phosphate medium in a 3.8 L baffled Fembach flask. Approximately 5 drops of antifoam were added to reduce foaming. The Fembach flask was shaken overnight at 30° C., to an optical density (OD₅₉₀ₙₘ) of approximately 40-60. This culture was used to inoculate a 10-L fermentor containing 8.0 L of Pichia Fermentation Tank Medium (see Table 2). A sufficient amount of the inoculum was added to give an initial OD₅₉₀ₙₘ in the fermentation tank of approximately 0.25. The temperature of the fermentation was controlled at 30° C. The culture was agitated using an impeller rotation rate of 1000 rpm, and was aerated at 16.7 L/min. The pH of the fermentation culture was maintained with additions of 2M phosphoric acid and 14.8 M ammonium hydroxide. During the initial batch phase of the fermentation the culture pH was maintained at 4.5. Antifoam was added as needed.

After approximately 15-19 hours of batch growth, the glycerol in the medium had been exhausted as evidenced by a rapid rise in the dissolved oxygen (DO) level in the fermentation culture. The rise in dissolved oxygen level triggered the initiation of the pre-induction phase of the culture, in which a glycerol feed was added at a controlled rate to maintain the DO level at 25% of saturation (with the limitation that the feed could not exceed 120 mL/hr). The glycerol feed, consisting of 50% glycerol and 1.2% PTM1 Trace Minerals with Biotin (Table 3), was continued for 3-6 hours.

Initiation of the induction phase of the fermentation entailed terminating the glycerol feed, starting a methanol feed, and adjusting the culture pH to 6.0. The pH change was accomplished by addition of 14.8 M ammonium hydroxide over the course of 1-2 hours. The methanol feed consisted of methanol supplemented with 1.2% PTM1 Trace Minerals with Biotin. The maximum methanol feed rate was initially 20 ml/hr. It was increased to 60 ml/hr after 3 hours and increased to 100 ml/hr after an additional 1 hour. The maximum methanol feed rate remained at 100 ml/hr until harvest. The feed control was programmed to feed at less than the maximal rate if the DO level dropped below 25%.

Samples were taken from the fermentor periodically for analysis. As part of sampling during the induction phase, the methanol feed was turned off briefly and the time was measured for the DO to increase by 10%. This DO response time was used to gauge whether methanol was accumulating in the fermentor. Times greater than one minute would have indicated overfeeding of methanol to a degree which could be toxic to the cells, in which case the rate of the methanol feed would have been reduced.

Approximately 90 hours after inoculation, the fermentor was harvested. At harvest, the fermentor contents were chilled, and the culture pH was adjusted to 4.0 by addition of 2M phosphoric acid. The fermentation broth was then clarified by centrifugation and the supernatant was filtered and stored frozen until purification of the hVEGF₁₂₁ dimer product was initiated.

TABLE 2
Pichia Fermentation Tank Medium

| Ingredient | Amount | |
|---|---|---|
| H₂O | 7 | L |
| 85% H₃PO₄ | 67.2 | mL |
| CaCl₂.2H₂O | 8.64 | g |
| K₂SO₄ | 68.80 | g |
| MgSO₄.7 H₂O | 56.16 | g |
| KOH | 15.6 | g |
| Peptone (Difco) | 80.0 | g |
| Adjust pH to 4.5 (with NaOH) then add | | |
| Glycerol | 180.0 | g |
| Adjust volume to 8.0 L, sterilize in fermentor, | | |
| cool to 30° C., then add | | |
| PTM1 Trace Minerals with Biotin (Table 2) | 32.0 | mL |
| 0.20 g/L Biotin | 64.0 | mL |

TABLE 3
PTM1 Trace Minerals with Biotin

| Ingredient | Amount | |
|---|---|---|
| CuSO₄.5H₂O | 6.00 | g |
| NaI | 0.08 | g |
| MnSO₄.H₂O | 3.00 | g |
| Na₂MoO₄.2H₂O | 0.20 | g |
| H₃BO₃ | 0.02 | g |
| CoCl₂.6H₂O | 0.91 | g |
| ZnCl₂ | 20.00 | g |
| FeCl₃.6H₂O | 20.78 | g |
| H₂SO₄ | 5.00 | mL |
| Biotin | 0.2 | g |
| H₂O | Up to 1.00 | L |

C. Purification of P. pastoris-derived hVEGF₁₂₁ N75Q Dimers

The filtered supernatant from the fermentation was first subjected to chromatography at pH 4.0 on SP-Sepharose (SP-Streamline, Pharmacia, Piscataway, N.J.) equilibrated in 50 mM sodium phosphate at either pH 3 or pH 4. After the supernatant was loaded on the column, the column was washed with equilibration buffer containing 0.2 M NaCl. The VEGF₁₂₁ N75Q product bound to the column was eluted with equilibration buffer containing 1.0 M NaCl. Alternatively, a gradient of 0.4 M -1.0 M NaCl in equilibration buffer was used for VEGF₁₂₁ elution. The eluate was adjusted to 1.2 M ammonium sulfate, 50 mM sodium phosphate, pH 7.0, and was loaded onto an Octyl-Sepharose Fast Flow column (Pharmacia) that had been equilibrated with 50 mM sodium phosphate, pH 7.0, 1.2 M ammonium sulfate. After a wash with column equilibration buffer, proteins bound to the column were eluted with a gradient of 1.2 M to 0 M ammonium sulfate in 50 mM sodium phosphate, pH 7.0. Fractions from the column elution were analyzed by SDS-PAGE followed by Coomassie staining to identify fractions containing the VEGF₁₂₁ product. The desired fractions were pooled and adjusted to 20 mM Tris, pH 7.4, 0.3 M NaCl, and were then loaded onto a [Zn²⁺]-Chelating Sepharose Fast Flow column (Pharmacia) equilibrated with 20 mM Tris, pH 7.4, 0.3 M NaCl. The column was washed with the column equilibration buffer, and bound proteins were eluted with an imidazole gradient (0-60 mM) in 20 mM Tris, pH 7.4, 0.3 M NaCl. Fractions shown by SDS-PAGE to contain VEGF₁₂₁ were pooled, concentrated in a stirred cell using a YM5 membrane, and then loaded onto a Vydac C4 preparative-scale reverse-phase HPLC column (The Separations Group, Hesperia, Calif.) equilibrated in 23.5% acetonitrile, 0.1% trifluoroacetic acid. Bound proteins were eluted with an acetonitrile gradient (23.5-33.4%) in 0.1% trifluoroacetic acid. The main protein peak in the elution profile was collected manually, lyophilized to dryness, resuspended in phosphate-buffered saline (pH 7.4), sterilized by filtration through a 0.22 μm filter, and stored frozen. Other protein peaks seen in the elution were also in some cases collected for analysis.

D. Analysis of hVEGF₁₂₁ N75Q Product

Amino-terminal sequencing indicated that 93-97% of the product initiated with the glutamic acid residue at position 5 of the native VEGF₁₂₁ sequence; that is, the majority of the product was missing the first 4 amino acids of the expected product. Small amounts (0.3-2.1%) of the product initiated with residue 6 (glycine), residue 7 (glycine), residue 8 (glycine), residue 11 (histidine), residue 12 (histidine), or residue 18 (methionine). Mass spectrometry analysis demonstrated that the product was dimeric but was also missing residue 121 (arginine). Thus, the majority of the final product from P. pastoris was made up of dimers consisting of monomers 116 residues in length.

The mass spectrometry data also indicated that some of the minor peaks collected from the final step of the purification contained either two additional cysteine moieties, or an additional cysteine moiety plus a glutathione moiety, presumably disulfide-bonded to the cysteine at position 116 in the VEGF₁₂₁ monomer subunits. However, no such additional cysteines or cysteine-containing peptides were seen on the major VEGF₁₂₁ product obtained from P. pastoris. These conclusions were confirmed by Glu-C digestion of the various products, followed by mass spectrometry analysis and/or sequencing of the products. These analyses confirmed that in the major product peak, the position 116 cysteine in each monomer subunit is paired with the other Cys-116 in the VEGF dimer, forming a third interchain disulfide bond.

### Example 4

### Selective Reduction of Cys-116 in P. pastoris-derived hVEGF₁₂₁ N75Q Dimers, and Demonstration of Instability of Resulting Product

A. Reduction of Cysteines at Residue Position 116 with Dithiotreitol (DTT)

Approximately 880 μg of hVEGF₁₂₁ N75Q (main product peak material, prepared as described in Example 3 above) were incubated with 1.6 mM DTT in 0.4 mL phosphate-buffered saline for 60 minutes at room temperature. The molar ratio of DTT to VEGF monomer in this mixture was thus 10 to 1. The reduction reaction was stopped by the addition of 0.1% trifluoroacetic acid to 0.05% (v/v) final concentration. The reaction was loaded onto a 5μ C4 250 mm×4.6 mm reverse-phase HPLC column (YMC Co, Kyoto, Japan) that was heated at 40° C. and equilibrated with 30% acetonitrile in 0.1% trifluoroacetic acid. Bound material was then eluted with a gradient of acetonitrile (30% to 35%) in 0.1% trifluoroacetic acid, at a flow rate of 1 mL/min. Under these conditions, the starting (non-reduced) P. pastoris-derived hVEGF₁₂₁ N75Q material eluted at about 24 minutes. The incubation with DTT generated several products, including one that eluted at about 10 minutes in the gradient (corresponding to about 40% of the total material eluted from the column). This peak was collected and lyophilized to dryness.

To confirm that the 10-minute peak material represented VEGF dimer product that was selectively reduced at Cys-116, three analyses were performed. First, an aliquot of the material was subjected to liquid chromatography-coupled mass spectrometry (LC-MS), which showed a mass of 27,111—consistent with the expected mass of partially-reduced 5-120 hVEGF₁₂₁ N75Q dimer. Second, titration of freshly-resuspended 10-minute peak material with 5,5′-dithiobis-(2-nitrobenzoic acid) (DTNB) indicated that two free sulfhydryl groups were present per dimer molecule. Third, an additional sample of the lyophilized material was resuspended in 0.15 mL of degassed 50 mM Tris, 150 mM NaCl, 5 mM EDTA, 10 mM iodoacetic acid, pH 8.5. The mixture was protected from light and incubated at room temperature for 2 hours. Under these conditions, the iodoacetic acid reacts with free sulfhydryl groups, but will not break disulfide bonds that are already present in a protein. The carboxymethylation reaction was stopped by applying the mixture to a NAP-5 gel filtration column (Pharmacia) that was equilibrated and eluted with phosphate-buffered saline. LC-MS analysis of an aliquot of the resulting protein showed a mass of 27,228.8, consistent with the presence of two carboxymethylations per dimer. The remaining iodoacetamide-treated material was then digested with the endopeptidase Glu-C, and the digestion products were subjected to amino-terminal sequencing. In the P. pastoris-derived 5-120 VEGF₁₂₁ dimer product, Glu-C cleaved after the glutamic acid residues at VEGF₁₂₁ residue positions 13 and 114. Three cleavage products were therefore generated, one of which represented residues 115-120. Hence, the state of the cysteine at position 116 was revealed in the second cycle of the sequencing. In this cycle, there was quantitative recovery of carboxymethylated cysteine, with no cystine or unmodified cysteine observed. The results thus confirmed that essentially all of the partially-reduced VEGF had contained two free sulfhydryl groups, one at each monomer position 116, prior to the carboxymethylation reaction.

B. Stability Test of Partially-Reduced VEGF₁₂₁ Dimer

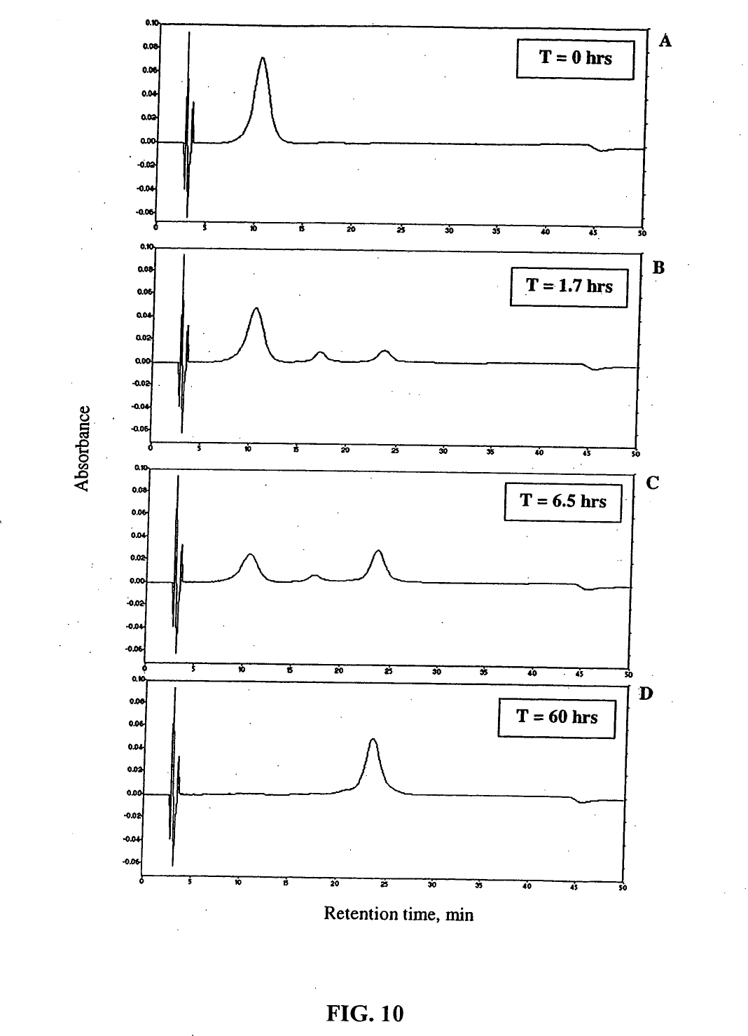

The partially-reduced VEGF (lyophilized 10-minute peak material isolated from YMC C4 column) was resuspended in degassed phosphate-buffered saline, and an aliquot was immediately reinjected onto the YMC C4 column. Essentially 100% of the resuspended protein eluted as a peak at the 10-minute point (FIG. 10A). The resuspended material was then incubated at 37° C., and additional aliquots were taken at various times for C4 HPLC analysis. The chromatography demonstrated that the partially-reduced VEGF rapidly underwent conversion. For example, as shown in FIG. 10C, after 6.5 hours of incubation at 37° C. only about 45% of the protein in the reaction continued to elute at the 10-minute position in the elution gradient. An additional 45% of the protein now eluted at approximately 24 minutes, with some material also eluting at about 17 minutes. At the end of the 6.5 hours of incubation at 37° C., the reaction was set at room temperature for two days. C4 reverse-phase HPLC analysis of a sample taken at that point showed that essentially no starting material (eluting at 10 minutes) remained in the mix, and virtually all of the protein was now eluting at approximately 24 minutes (FIG. 10D).

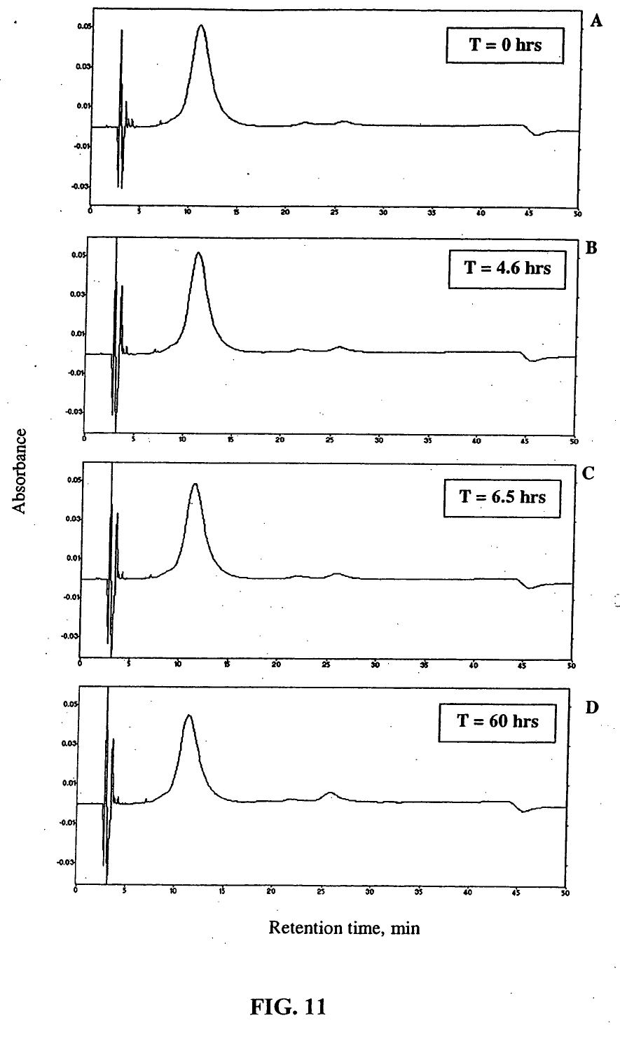

A similar stability experiment is carried out using hVEGF₁₂₁ dimeric protein in which two additional cysteines were present in the molecule, disulfide bonded to the two Cys-116 residues in the dimer. Under the same C4 reverse-phase HPLC conditions as used in the experiment described in the previous paragraph, this material eluted at about 11.5 minutes in the elution gradient (FIG. 11A). As shown in FIGS. 11B-11D, incubation of this material in phosphate-buffered saline at 37° C. for 6.5 hours, followed by incubation for 2 days at room temperature, produced little if any noticable change in the molecule, at least as judged by reverse-phase HPLC analysis.

### Example 5

### HUVE Cell Proliferation Assay—BrdU ELISA

Assay

96-well plates were coated with human fibronectin (Sigma, 1 μg/100 μl/well) in phosphate-buffered saline (PBS). The plates were incubated at room temperature for 45 minutes, the fibronectin solution was aspirated, and the plates were dried for 20-30 minutes open to air. Cells (HUVEC, Clonetics) were then plated at 10000 cells/100 μl/well in human endothelial cell serum free medium (Gibco)+2% fetal bovine serum (FBS), leaving the first column of wells in each 96-well plate cell-free to act as a blank. The cells were incubated at 37° C., 5% CO₂ overnight (18-24 hours). The medium was changed to 100 μl/well serum-free medium+1% FBS, and the plates were incubated at 37° C., 5% CO₂ for 24 hours to allow the cells to quiesce.

VEGF₁₂₁ standards and the samples to be tested were diluted serially 1:3 in serum-free medium+0.1% human serum albumin (HSA, Sigma). 10 μl of the dilutions were added to the wells, which were incubated at 37° C., 5% CO₂ for 24 hours. Bromodeoxyuridine (BrdU) solution from the cell proliferation ELISA kit (Boehringer Mannheim) was diluted 1:100 with Gibco serum-free medium, and 12 μl of this solution was added to each well. The plates were then incubated at 37° C., 5% CO₂ for 4-5 hours. BrdU was omitted for the wells used as background control.

After 4-5 hours incubation, the medium was aspirated, 200 μl FixDeNat solution from the ELISA was added to each well, and the plates were incubated at room temperature for 30 minutes. FixDeNat was thoroughly aspirated, 100 μl anti-BrdU-POD (anti-BrdU-peroxidase) antibody solution from the kit was added from the kit to each well (1:100 dilution of anti-BrdU-POD into PBS+0.05% Tween20+0.5% HSA), and the plates were incubated at room temperature for 90 minutes. Wells were washed four times with 300 μl/well of PBS+0.05% Tween20, and 100 μl TMB substrate was added. This was followed by incubation for 20-30 minutes until the color was sufficient for calorimetric reading, whereupon 50 μl sulfuric acid (5N) was added, and colorimetric reading was performed at an absorbance of 450 nm.

Results

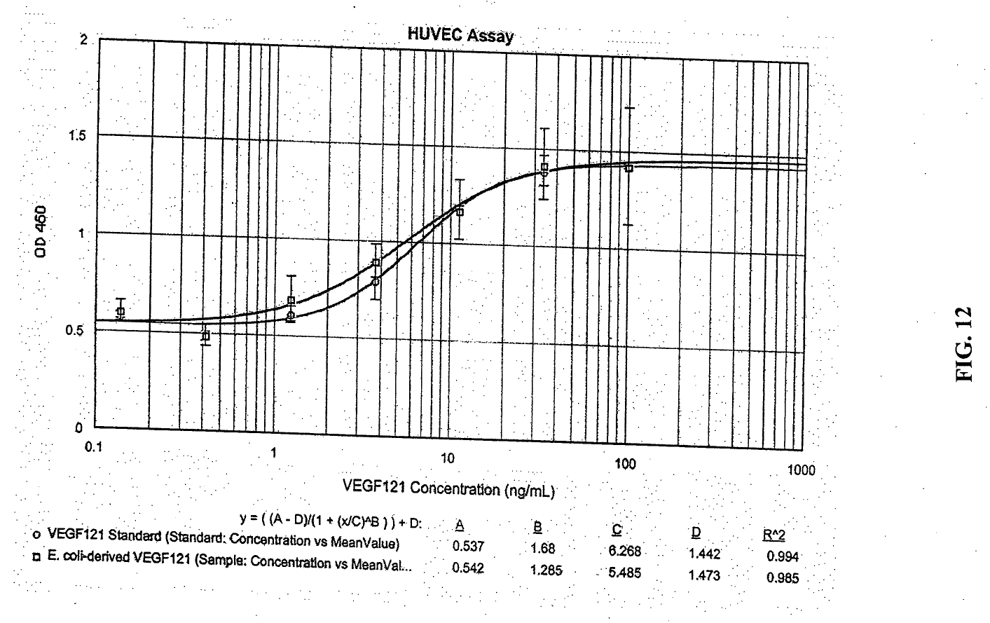

The results are shown in FIG. 12. The graph depicts the amount of DNA synthesis that was stimulated in response to serial dilutions of Pichia-derived N75Q VEGF₁₂₁ (VEGF standard; primarily consisting of molecules containing three interchain disulfide bonds) vs. E. coli-derived VEGF₁₂₁ (primarily consisting of molecules with only two interchain disulfide bonds, with additional extraneous cysteines disulfide-bonded to the Cys-116 residues). The X axis of the graph represents the final concentration of added growth factor in the assay wells, expressed as ng/ml. The Y axis represents the optical density recorded in each well after use of the BrdU kit (Boehringer Mannheim) to detect incorporated bromodeoxyuridine at the end of the assay.

The ED₅₀ (effective dose of growth factor needed to achieve a half-maximal proliferation response) for the VEGF₁₂₁ standard was 6.27 ng/ml, while E. coli-derived VEGF₁₂₁ showed an ED₅₀ of 5.48 ng/ml. Thus, the E. coli-derived VEGF₁₂₁ in this assay was as potent as, if not slightly more potent than, the VEGF₁₂₁ standard in promoting DNA synthesis.

```
<160> NUMBER OF SEQ ID NOS: 3
<210> SEQ ID NO 1
<211> LENGTH: 444
<212> TYPE: DNA
<213> ORGANISM: Homo sapiens
<220> FEATURE:
<221> NAME/KEY: CDS
<222> LOCATION: (1)...(441)
<400> SEQUENCE: 1
atg aac ttt ctg ctg tct tgg gtg cat tgg ag
#c ctt gcc ttg ctg ctc 48
Met Asn Phe Leu Leu Ser Trp Val His Trp Se
#r Leu Ala Leu Leu Leu
  1 5
# 10
# 15
tac ctc cac cat gcc aag tgg tcc cag gct gc
#a ccc atg gca gaa gga 96
Tyr Leu His His Ala Lys Trp Ser Gln Ala Al
#a Pro Met Ala Glu Gly
                          20
# 25
# 30
gga ggg cag aat cat cac gaa gtg gtg aag tt
#c atg gat gtc tat cag 144
Gly Gly Gln Asn His His Glu Val Val Lys Ph
#e Met Asp Val Tyr Gln
                  35
# 40
# 45
cgc agc tac tgc cat cca atc gag acc ctg gt
#g gac atc ttc cag gag 192
Arg Ser Tyr Cys His Pro Ile Glu Thr Leu Va
#l Asp Ile Phe Gln Glu
          50
# 55
# 60
tac cct gat gag atc gag tac atc ttc aag cc
#a tcc tgt gtg ccc ctg 240
Tyr Pro Asp Glu Ile Glu Tyr Ile Phe Lys Pr
#o Ser Cys Val Pro Leu
  65
# 70
# 75
# 80
atg cga tgc ggg ggc tgc tgc aat gac gag gg
#c ctg gag tgt gtg ccc 288
Met Arg Cys Gly Gly Cys Cys Asn Asp Glu Gl
#y Leu Glu Cys Val Pro
                                  85
# 90
# 95
act gag gag tcc aac atc acc atg cag att at
#g cgg atc aaa cct cac 336
Thr Glu Glu Ser Asn Ile Thr Met Gln Ile Me
#t Arg Ile Lys Pro His
                        100
# 105
# 110
caa ggc cag cac ata gga gag atg agc ttc ct
#a cag cac aac aaa tgt 384
Gln Gly Gln His Ile Gly Glu Met Ser Phe Le
#u Gln His Asn Lys Cys
                115
# 120
# 125
gaa tgc aga cca aag aaa gat aga gca aga ca
#a gaa aaa tgt gac aag 432
Glu Cys Arg Pro Lys Lys Asp Arg Ala Arg Gl
#n Glu Lys Cys Asp Lys
        130
# 135
# 140
ccg agg cgg tga
#
#
# 444
Pro Arg Arg
145
<210> SEQ ID NO 2
<211> LENGTH: 147
<212> TYPE: PRT
<213> ORGANISM: Homo sapiens
<400> SEQUENCE: 2
Met Asn Phe Leu Leu Ser Trp Val His Trp Se
#r Leu Ala Leu Leu Leu
  1 5
# 10
# 15
Tyr Leu His His Ala Lys Trp Ser Gln Ala Al
#a Pro Met Ala Glu Gly
                        20
# 25
# 30
Gly Gly Gln Asn His His Glu Val Val Lys Ph
#e Met Asp Val Tyr Gln
                35
# 40
# 45
Arg Ser Tyr Cys His Pro Ile Glu Thr Leu Va
#l Asp Ile Phe Gln Glu
        50
# 55
# 60
Tyr Pro Asp Glu Ile Glu Tyr Ile Phe Lys Pr
#o Ser Cys Val Pro Leu
65
#70
#75
#80
Met Arg Cys Gly Gly Cys Cys Asn Asp Glu Gl
#y Leu Glu Cys Val Pro
                                85
# 90
# 95
Thr Glu Glu Ser Asn Ile Thr Met Gln Ile Me
#t Arg Ile Lys Pro His
                        100
# 105
# 110
Gln Gly Gln His Ile Gly Glu Met Ser Phe Le
#u Gln His Asn Lys Cys
                115
# 120
# 125
Glu Cys Arg Pro Lys Lys Asp Arg Ala Arg Gl
#n Glu Lys Cys Asp Lys
        130
# 135
# 140
Pro Arg Arg
145
<210> SEQ ID NO 3
<211> LENGTH: 366
<212> TYPE: DNA
<213> ORGANISM: Homo sapiens
<220> FEATURE:
<221> NAME/KEY: CDS
<222> LOCATION: (1)...(363)
<400> SEQUENCE: 3
gca ccc atg gca gaa gga gga ggg cag aat ca
#t cac gaa gtg gtg aag 48
Ala Pro Met Ala Glu Gly Gly Gly Gln Asn Hi
#s His Glu Val Val Lys
                                  5
# 10
# 15
ttc atg gat gtc tat cag cgc agc tac tgc ca
#t cca atc gag acc ctg 96
Phe Met Asp Val Tyr Gln Arg Ser Tyr Cys Hi
#s Pro Ile Glu Thr Leu
                        20
# 25
# 30
gtg gac atc ttc cag gag tac cct gat gag at
#c gag tac atc ttc aag 144
Val Asp Ile Phe Gln Glu Tyr Pro Asp Glu Il
#e Glu Tyr Ile Phe Lys
                35
# 40
# 45
cca tcc tgt gtg ccc ctg atg cga tgc ggg gg
#c tgc tgc aat gac gag 192
Pro Ser Cys Val Pro Leu Met Arg Cys Gly Gl
#y Cys Cys Asn Asp Glu
        50
# 55
# 60
ggc ctg gag tgt gtg ccc act gag gag tcc aa
#c atc acc atg cag att 240
Gly Leu Glu Cys Val Pro Thr Glu Glu Ser As
#n Ile Thr Met Gln Ile
65
#70
#75
#80
atg cgg atc aaa cct cac caa ggc cag cac at
#a gga gag atg agc ttc 288
Met Arg Ile Lys Pro His Gln Gly Gln His Il
#e Gly Glu Met Ser Phe
                                85
# 90
# 95
cta cag cac aac aaa tgt gaa tgc aga cca aa
#g aaa gat aga gca aga 336
Leu Gln His Asn Lys Cys Glu Cys Arg Pro Ly
#s Lys Asp Arg Ala Arg
                        100
# 105
# 110
caa gaa aaa tgt gac aag ccg agg cgg tga
#
# 366
Gln Glu Lys Cys Asp Lys Pro Arg Arg
                115
# 120
```

What is claimed is:

1. A process for preparing a vascular endothelial growth factor (VEGF) dimer comprising:
providing transformed host bacterial cells, wherein the transformed host bacterial cells comprise an exogenous nucleic acid encoding an amino acid sequence of a VEGF monomer operably linked to a promoter, wherein the amino acid sequence has at least about 90% sequence identity with amino acids 11 to 116 of SEQ ID NO: 1 and wherein the amino acid sequence is extended by a Met-(AA)ₙ- sequence at the amino terminus (N-terminus), wherein Met stands for methionine, n is 1-7, and AA represents identical or different amino acids, where at least one of the AA amino acids, or a combination of two or more of the AA amino acids, is capable of retarding proteolytic degradation of the mature N-terminus of the VEGF dimer by the bacterial host cell, and the amino acid sequence retains a cysteine (Cys) at or corresponding to position 116 of SEQ ID NO: 1 (Cys-116);
culturing said host cells under conditions suitable for expression of said VEGF monomer, whereby a first VEGF monomer and a second VEGF monomer are produced;
forming the VEGF dimer from the first and second VEGF monomers; and
recovering said VEGF dimer.

2. The process of claim 1, wherein n is 1.

3. The process of claim 2, wherein AA represents an amino acid selected from the group consisting of lysine (Lys) and arginine (Arg) residues.

4. The process of claim 3, wherein AA represents a lysine (Lys) residue.

5. The process of claim 1, further comprising the step of purifying said VEGF dimers.

6. The process of claim 5, further comprising the removal of the N-terminal Met(AA)ₙ- sequence following at least partial purification.

7. The process of claim 6, wherein removal is performed by enzymatic digestion.

8. The process of claim 7, wherein diaminopeptidase is used to perform the enzymatic digestion.

9. The process of claim 1, wherein at least about 95% of said VEGF dimers are devoid of an N-terminal methionine residue.

10. The process of claim 1, further comprising the step of refolding said VEGF dimers.

11. The process of claim 10, wherein refolding is performed in a refolding buffer comprising cysteine and cystine in amounts and in a ratio to each other sufficient to produce the desired mixture of VEGF dimers.

12. A process for producing a vascular endothelial growth factor (VEGF) dimer composed of two VEGF monomers, in which each monomer comprises amino acids 11 to 116 of SEQ ID NO: 1, or comprises an amino acid sequence having at least about 90% sequence identity with amino acids 11 to 116 of SEQ ID NO: 1, and retaining a cysteine (Cys) at a position corresponding to position 116 of SEQ ID NO: 1 (Cys-116), where Cys-116 of each monomer is disulfide bonded to an additional extraneous Cys, comprising the steps of:
providing transformed bacterial host cells comprising a species of exogenous nucleic acid encoding a promoter operably linked to a polypeptide of SEQ ID NO: 1 extended by a Met-(AA)ₙ- sequence at the amino terminus (N-terminus), wherein Met stands for methionine, n is 1-7, and AA represents identical or different amino acids, wherein at least one of the AA amino acids, or a combination of two or more of the AA amino acids, is capable of blocking the proteolytic degradation of the mature N-terminus of the VEGF polypeptides by the bacterial host cell;
culturing said bacterial host cells under conditions suitable for expression of said exogenous nucleic acid and the synthesis of said N-terminally-extended VEGF monomers, and recovering said VEGF dimer.

13. The process of claim 12, wherein n is 1.

14. The process of claim 13, wherein AA represents an amino acid selected from the group consisting of lysine (Lys) and arginine (Arg) residues.

15. The process of claim 14, wherein AA represents a lysine (Lys) residue.

16. The process of claim 12, further comprising the step of purifying said VEGF dimer.

17. The process of claim 16, further comprising the removal of the N-terminal Met(AA)ₙ- sequence following at least partial purification.

18. The process of claim 17, wherein removal is performed by enzymatic digestion.

19. The process of claim 18, wherein at least about 95% of said VEGF dimers are devoid of an N-terminal methionine residue.

20. The process of claim 12, additionally comprising the step of refolding said VEGF dimer.

21. The process of claim 14, additionally comprising the step of refolding said VEGF dimer.

22. The process of claim 17, additionally comprising the step of refolding said VEGF dimer.

23. The process of claim 22, wherein refolding is performed in a refolding buffer comprising cysteine and cystine.

24. A process for preparing a vascular endothelial growth factor (VEGF) dimer comprising:
providing host cells, wherein the host cells comprise an exogenous nucleic acid encoding an amino acid sequence of a VEGF monomer operably linked to a promoter, wherein the amino acid sequence has at least about 90% sequence identity with amino acids 11 to 116 of SEQ ID NO: 1, retains a cysteine (Cys) at or corresponding to position 116 of SEQ ID NO: 1 (Cys-116), and wherein at least one monomer has an Asn-to-Glu amino acid substitution at or corresponding to position 75 of SEQ ID NO: 1;
culturing said host cells under conditions suitable for expression of said VEGF monomer, whereby a first VEGF monomer and a second VEGF monomer are produced;
forming the VEGF dimer from the first and second VEGF monomers; and
recovering said VEGF dimer.

25. The process of claim 24, wherein each monomer comprises amino acids 1 to 120 of SEQ ID NO: 1.

26. The process of claim 24, wherein monomer comprises amino acids 1 to 121 of SEQ ID NO: 1.

27. The process of claim 24, wherein at least about 95% of said VEGF dimers are devoid of an N-terminal methionine residue.

28. The process of claim 24, wherein the Cys residue corresponding to Cys-116 of SEQ ID NO: 1 of each monomer is disulfide bonded to an extraneous Cys.

29. The process of claim 24, wherein the Cys residue corresponding to Cys-116 of SEQ ID NO: 1 of the two monomers are interconnected with an interchain disulfide bond.

30. The process of claim 24, wherein the Cys residue corresponding to Cys-116 of SEQ ID NO: 1 of one or both monomers is not reduced.

31. The process of claim 24, additionally comprising the step of purifying said dimers.

32. The process of claim 24, wherein said transformed host cells are bacterial cells.

33. The process of claim 32, wherein said bacterial cells are E. coli cells.

34. The process of claim 32, wherein the exogenous nucleic acid encodes a polypeptide of SEQ ID NO: 1 extended by a Met-(AA)ₙ- sequence at the amino terminus (N-terminus), wherein Met stands for methionine, n is 1-7, and AA represents identical or different amino acids, where at least one of the AA amino acids, or a combination of two or more of the AA amino acids, is capable of retarding proteolytic degradation of the mature N-terminus of the VEGF dimer by the bacterial host cell.

35. The process of claim 34, wherein n is 1.

36. The process of claim 35, wherein AA represents an amino acid selected from the group consisting of lysine (Lys) and arginine (Arg) residues.

37. The process of claim 36, wherein AA represents a lysine (Lys) residue.

38. The process of claim 34, further comprising the step of purifying said VEGF dimers.

39. The process of claim 38, further comprising the removal of the N-terminal Met(AA)ₙ- sequence following at least partial purification.

40. The process of claim 39, wherein removal is performed by enzymatic digestion.

41. The process of claim 32, further comprising the step of refolding said VEGF dimers.

42. The process of claim 41, wherein refolding is performed in a refolding buffer comprising cysteine and cystine in amounts and in a ratio to each other sufficient to produce the desired mixture of VEGF dimers.
